(19) United States
(12) Patent Application Publication
(10) Pub. No.: US 2023/0408506 A1
(43) Pub. Date: Dec. 21, 2023

(54) SENSORS, SYSTEMS AND METHODS FOR DETECTING ANALYTES

(71) Applicant: LACRISCIENCES, LLC, Washington, DC (US)

(72) Inventor: Chris D. Geddes, Bel Air, MD (US)

(21) Appl. No.: 18/248,864

(22) PCT Filed: Oct. 12, 2021
(86) PCT No.: PCT/US2021/054530

§ 371 Date: Apr. 12, 2023

Related U.S. Application Data
(60) Provisional application No. 63/090,609, filed on Oct. 12, 2020.

(51) Int. Cl. G01N 33/543; G01N 33/569; G01N 21/65

(52) U.S. Cl. CPC G01N 33/54373; G01N 33/56983; G01N 21/658; G01N 2333/165; G01N 2469/20

(57) ABSTRACT

Sensors, systems and methods for detecting analytes in a sample are provided. Aspects of the subject methods include contacting a sensing surface of a sensor with a sample, and generating one or more data sets over a time interval, wherein the data sets are used to determine the presence or absence of a member of a binding pair in the sample. The subject methods find use in determining the presence or absence of one or more analytes in a sample, such as a biological sample (e.g., blood), and in the diagnosis and/or monitoring of various diseases and disorders, such as, e.g., infection with a virus.

### CROSS-REFERENCE TO RELATED APPLICATIONS

This application claims priority benefit of the filing date of U.S. Provisional Patent Application Ser. No. 63/090,609, filed on Oct. 12, 2020, the disclosure of which is incorporated by reference herein in its entirety.

### FIELD OF THE INVENTION

The present invention relates to sensors, systems and methods for detecting analytes in a sample, such as a biological sample (e.g., blood).

### BACKGROUND OF THE INVENTION

Direct detection of analytes in a sample remains a major challenge in the field of bioanalytical chemistry, and systems that can monitor binding interactions between molecules, such as biomolecules, in real time, continue to gain interest. While some systems allow for detection of analytes without purifying the substances involved, most still require comparison to one or more calibration solutions having known concentrations of the target analyte being detected, and often require large-scale laboratory equipment to complete the analysis. As such, many detection modalities must be conducted in a laboratory setting and are not amenable to portable applications where an end user can determine a result in real time. Accordingly, there remains a need for improved analyte detection systems that can meet these and other needs.

### SUMMARY

Sensors, systems and methods for detecting analytes in a sample are provided. Aspects of the subject methods include contacting a sensing surface of a sensor with a sample, and generating one or more data sets over a time interval, wherein the data sets are used to determine the presence or absence of a member of a binding pair in the sample. The subject methods find use in determining the presence or absence of one or more analytes in a sample, such as a biological sample (e.g., blood), and in the diagnosis and/or monitoring of various diseases and disorders, such as, e.g., infection with a virus.

Aspects of the invention include sensors comprising: a sensing surface comprising a coated region, wherein the coated region comprises a first member of a binding pair immobilized thereon; and wherein the sensor is configured to: direct a first optical signal to interact with the sensing surface over a first range of incident angles; and direct a second optical signal to interact with the sensing surface over a second range of incident angles, wherein the first range of incident angles is different from the second range of incident angles.

In some embodiments, the sensor comprises a plurality of facets. In some embodiments, the sensor has a frustoconical, concave shape. In some embodiments, the sensor comprises a plurality of facets on an internal surface and a plurality of facets on an external surface. In some embodiments, the sensor comprises 2 facets on the internal surface and 4 facets on the external surface.

In some embodiments, the sensing surface is disposed on a central portion of the sensor. In some embodiments, the sensing surface further comprises a non-coated region. In some embodiments, the coated region comprises a semitransparent film that comprises a noble metal. In some embodiments, the noble metal is selected from the group consisting of: gold, silver, aluminum, platinum, palladium, or any combination thereof. In some embodiments, the semitransparent film has a thickness that ranges from about 0.5 nm to about 200 nm. In some embodiments, the semitransparent film has a thickness of about 45 to about 50 nm. In some embodiments, the coated region comprises an adhesion layer that is disposed between the sensor and the semitransparent film. In some embodiments, the adhesion layer has a thickness that ranges from about 0.5 nm to about 200 nm. In some embodiments, the adhesion layer has a thickness that ranges from about 45 nm to about 50 nm. In some embodiments, the adhesion layer comprises a material selected from the group consisting of: chromium, titanium dioxide, titanium monoxide, silicon dioxide, silicon monoxide, or any combination thereof. In some embodiments, the adhesion layer has an index of refraction that is different from an index of refraction of the sensor.

In some embodiments, the first range of incident angles spans about 40 to 45 degrees. In some embodiments, the sensor is configured to direct the first optical signal to interact with the sensing surface at an angle of about 42 degrees. In some embodiments, the second range of incident angles spans about 62 to 67 degrees. In some embodiments, the sensor is configured to direct the second optical signal to interact with the sensing surface at an angle of about 64 degrees.

In some embodiments, the binding pair is an antigen-antibody binding pair, and wherein the first member of the binding pair is the antigen. In some embodiments, the binding pair is an antigen-antibody binding pair, and wherein the first member of the binding pair is the antibody. In some embodiments, the antigen is a viral protein antigen. In some embodiments, the viral protein antigen is selected from the group consisting of: a viral membrane protein, a viral envelop protein, or a viral nucleoprotein. In some embodiments, the viral protein antigen is a coronavirus spike protein. In some embodiments, the coronavirus spike protein is a SARS-CoV-2 spike protein. In some embodiments, the SARS-CoV-2 spike protein is an S1 or an S2 subunit protein.

Aspects of the invention include systems comprising: (i) a sensor as described herein; and (ii) an optical chassis comprising: an optical signal generating component; a detection component; a processor; a controller; and a computer-readable medium comprising instructions that, when executed by the processor, cause the controller to: direct an optical signal having a first wavelength to interact with the sensing surface over the first range of incident angles to generate a first surface plasmon resonance (SPR) signal; generate an image of the first SPR signal using the detection component; determine a pixel position of a minimum value of the first SPR signal on the generated image to generate an SPR reference value; direct an optical signal having the first wavelength to interact with the sensing surface over the second range of incident angles to generate a second SPR signal; generate a series of images of the second SPR signal over a first time interval using the detection component; determine a series of pixel positions that correspond to a minimum value of the second SPR signal over the first time interval; determine a rate of change of the series of pixel positions that corresponds to the minimum value of the second SPR signal over the first time interval; determine a plateau value of the second SPR signal based on the rate of change of the series of pixel positions that corresponds to the minimum value of the second SPR signal over the first time interval to generate an SPR test value; and compare the SPR test value to the SPR reference value.

In some embodiments, the computer-readable medium further comprises instructions that, when executed by the processor, cause the controller to: direct an optical signal having a second wavelength to interact with the sensing surface over the first range of incident angles to generate a third SPR signal; generate an image of the third SPR signal using the detection component; determine a pixel position of a minimum value of the third SPR signal on the generated image; and combine the pixel position of the minimum value of the first SPR signal and the pixel position of the minimum value of the third SPR signal to generate the SPR reference value.

In some embodiments, the computer-readable medium further comprises instructions that, when executed by the processor, cause the controller to: direct an optical signal having a second wavelength to interact with the sensing surface over the second range of incident angles to generate a fourth SPR signal; generate a series of images of the fourth SPR signal over a second time interval using the detection component; determine a series of pixel positions that corresponds to a minimum value of the fourth SPR signal over the second time interval; determine a rate of change of the series of pixel positions that corresponds to the minimum value of the fourth SPR signal over the second time interval; determine a plateau value of the fourth SPR signal based on the rate of change of the series of pixel positions that corresponds to the minimum value of the fourth SPR signal over the second time interval; and combine the plateau value of the second SPR signal and the plateau value of the fourth SPR signal to generate the SPR test value.

In some embodiments, the computer-readable medium further comprises instructions that, when executed by the processor, cause the controller to: direct an optical signal having a first wavelength to interact with the sensing surface over the first range of incident angles to generate a first critical angle signal; generate an image of the first critical angle signal using the detection component; and determine a pixel position of a maximum value of the first critical angle signal on the generated image to generate a critical angle reference value.

In some embodiments, the computer-readable medium further comprises instructions that, when executed by the processor, cause the controller to: direct an optical signal having a second wavelength to interact with the sensing surface over the first range of incident angles to generate a second critical angle signal; generate an image of the second critical angle signal using the detection component; determine a pixel position of a maximum value of the second critical angle signal on the generated image; and combine the pixel position of the maximum value of the first critical angle signal and the pixel position of the maximum value of the second critical angle signal to generate the critical angle reference value. In some embodiments, the sensor comprises a coated region and a non-coated region, and wherein the first and second critical angle signals are generated from the non-coated region. In some embodiments, the computer-readable medium further comprises instructions that, when executed by the processor, cause the controller to determine a pixel position corresponding to an internal reference feature. In some embodiments, the internal reference comprises an opto-mechanical reference feature. In some embodiments, the computer-readable medium further comprises instructions that, when executed by the processor, cause the controller to compare one or more generated values to a calibration data set.

In some embodiments, the first range of incident angles spans about 40 to 45 degrees. In some embodiments, the sensor is configured to direct the first optical signal to interact with the sensing surface at an angle of about 42 degrees. In some embodiments, the second range of incident angles spans about 62 to 67 degrees. In some embodiments, the sensor is configured to direct the second optical signal to interact with the sensing surface at an angle of about 64 degrees.

In some embodiments, the optical signal generating component comprises a laser or a light emitting diode (LED). In some embodiments, the laser or the LED emits visible or infrared light. In some embodiments, the laser or the LED emits light having a wavelength that ranges from about 400 to about 1,000 nm. In some embodiments, the laser or the LED is configured to emit light having a wavelength of about 855 nm. In some embodiments, the laser or the LED is configured to emit light having a wavelength of about 950 nm. In some embodiments, the optical chassis further comprises one or more optical signal manipulation components. In some embodiments, the detection component comprises an image sensor. In some embodiments, the image sensor is a charge coupled device (CCD) camera or a scientific complementary metal-oxide semiconductor (sCMOS) camera. In some embodiments, the image sensor is an active pixel sensor (APS).

In some embodiments, a system further comprises a plurality of retention fixtures that are configured to removably couple the sensor to the optical chassis. In some embodiments, a system further comprises an alignment component that is configured to align the sensor with the optical chassis. In some embodiments, the alignment component comprises a tapered centering component. In some embodiments, a system further comprises a plurality of kinematic mounting components. In some embodiments, the sensor is configured to be removably coupled to the optical chassis. In some embodiments, the system is a benchtop system. In some embodiments, the system is a hand-held system.

Aspects of the invention include methods for detecting the presence of a second member of a binding pair in a test sample, the methods comprising: contacting a sensing surface of a system as described herein with a reference fluid; directing an optical signal having a first wavelength to interact with the sensing surface over the first range of incident angles to generate a first surface plasmon resonance (SPR) signal; generating an image of the first SPR signal using the detection component; determining a pixel position of a minimum value of the first SPR signal on the generated image to generate an SPR reference value; contacting the sensing surface with a test sample; directing an optical signal having the first wavelength to interact with the sensing surface over the second range of incident angles to generate a second SPR signal; generating a series of images of the second SPR signal over a first time interval using the detection component; determining a series of pixel positions that correspond to a minimum value of the second SPR signal over the first time interval; determining a rate of change of the series of pixel positions that corresponds to the minimum value of the second SPR signal over the first time interval; determining a plateau value of the second SPR signal based on the rate of change of the series of pixel positions that corresponds to the minimum value of the second SPR signal over the first time interval to generate an SPR test value; and comparing the SPR test value to the SPR reference value to detect the presence of the second member of the binding pair in the test sample.

In some embodiments, a method further comprises: directing an optical signal having a second wavelength to interact with the sensing surface over the first range of incident angles to generate a third SPR signal while the sensing surface is in contact with the reference fluid; generating an image of the third SPR signal using the detection component; determining a pixel position of a minimum value of the third SPR signal on the generated image; and combining the pixel position of the minimum value of the first SPR signal and the pixel position of the minimum value of the third SPR signal to generate the SPR reference value.

In some embodiments, a method further comprises: directing an optical signal having a second wavelength to interact with the sensing surface over the second range of incident angles to generate a fourth SPR signal while the sensing surface is in contact with the test sample; generating a series of images of the fourth SPR signal over a second time interval using the detection component; determining a series of pixel positions that corresponds to a minimum value of the fourth SPR signal over the second time interval; determining a rate of change of the series of pixel positions that corresponds to the minimum value of the fourth SPR signal over the second time interval; determining a plateau value of the fourth SPR signal based on the rate of change of the series of pixel positions that corresponds to the minimum value of the fourth SPR signal over the second time interval; and combining the plateau value of the second SPR signal and the plateau value of the fourth SPR signal to generate the SPR test value.

In some embodiments, a method further comprises: directing an optical signal having a first wavelength to interact with the sensing surface over the first range of incident angles to generate a first critical angle signal while the sensing surface is in contact with the reference fluid; generating an image of the first critical angle signal using the detection component; and determining a pixel position of a maximum value of the first critical angle signal on the generated image to generate a critical angle reference value.

In some embodiments, a method further comprises: directing an optical signal having a second wavelength to interact with the sensing surface over the first range of incident angles to generate a second critical angle signal while the sensing surface is in contact with the reference fluid; generating an image of the second critical angle signal using the detection component; determining a pixel position of a maximum value of the second critical angle signal on the generated image; and combining the pixel position of the maximum value of the first critical angle signal and the pixel position of the maximum value of the second critical angle signal to generate the critical angle reference value.

In some embodiments, a method further comprises determining a pixel position corresponding to an internal reference feature. In some embodiments, the internal reference feature comprises an opto-mechanical reference feature. In some embodiments, the first range of incident angles spans about 40 to 45 degrees. In some embodiments, the sensor is configured to direct the first optical signal to interact with the sensing surface at an angle of about 42 degrees. In some embodiments, the second range of incident angles spans about 62 to 67 degrees. In some embodiments, the sensor is configured to direct the second optical signal to interact with the sensing surface at an angle of about 64 degrees.

In some embodiments, the images of the SPR signals are captured in a single image frame. In some embodiments, the images of the SPR signals and the images of the critical angle signals are captured in a single image frame.

In some embodiments, a method further comprises comparing one or more generated values to a calibration data set. In some embodiments, a method further comprises: comparing one or more generated values to an external environment parameter to generate an external environment corrected value; and comparing the external environment corrected value to a calibration data set. In some embodiments, the external environment parameter is selected from the group comprising: temperature, pressure, humidity, light, environmental composition, or any combination thereof.

In some embodiments, the optical signals having a first and a second wavelength are directed to interact with the sensing surface simultaneously. In some embodiments, the optical signals having a first and second wavelength are directed to interact with the sensing surface in a gated manner.

In some embodiments, the calibration data set is stored in a read-only memory of a processor of the system.

In some embodiments, the sample is a biological sample. In some embodiments, the biological sample comprises blood. In some embodiments, the reference fluid comprises water. In some embodiments, the reference fluid is air.

In some embodiments, the first time interval ranges from about 0.001 seconds to about 90 seconds. In some embodiments, the second time interval ranges from about 0.001 seconds to about 90 seconds.

Aspects of the invention include methods for determining an antibody isotype response in a subject, the methods comprising: contacting a sensing surface of a system as described herein with a reference fluid; directing an optical signal having a first wavelength to interact with the sensing surface over the first range of incident angles to generate a first surface plasmon resonance (SPR) signal; generating an image of the first SPR signal using the detection component; determining a pixel position of a minimum value of the first SPR signal on the generated image to generate an SPR reference value; contacting the sensing surface with a sample from the subject, wherein the binding pair is an antigen-antibody binding pair, wherein the first member of the binding pair is the antigen, and wherein the sample comprises a plurality of antibody isotypes that bind to the antigen; directing an optical signal having the first wavelength to interact with the sensing surface over the second range of incident angles to generate a second SPR signal; generating a series of images of the second SPR signal over a first time interval using the detection component; determining a series of pixel positions that correspond to a minimum value of the second SPR signal over the first time interval; determining a rate of change of the series of pixel positions that corresponds to the minimum value of the second SPR signal over the first time interval; determining a plateau value of the second SPR signal based on the rate of change of the series of pixel positions that corresponds to the minimum value of the second SPR signal over the first time interval to generate a first SPR test value; contacting the sensing surface with a stripping agent that removes at least one antibody isotype; directing an optical signal having the first wavelength to interact with the sensing surface over the second range of incident angles to generate a third SPR signal; generating a series of images of the third SPR signal over a second time interval using the detection component; determining a series of pixel positions that correspond to a minimum value of the third SPR signal over the second time interval; determining a rate of change of the series of pixel positions that corresponds to the minimum value of the third SPR signal over the second time interval; determining a plateau value of the third SPR signal based on the rate of change of the series of pixel positions that corresponds to the minimum value of the third SPR signal over the second time interval to generate a second SPR test value; comparing the first SPR test value, the second SPR test value, and the SPR reference value to determine the antibody isotype response in the subject.

Aspects of the invention include methods for determining a coronavirus exposure status in a patient, the methods comprising: contacting a sensing surface of a system as described herein with a reference fluid; directing an optical signal having a first wavelength to interact with the sensing surface over the first range of incident angles to generate a first surface plasmon resonance (SPR) signal; generating an image of the first SPR signal using the detection component; determining a pixel position of a minimum value of the first SPR signal on the generated image to generate an SPR reference value; contacting the sensing surface with a sample from the patient, wherein the binding pair is an antigen-antibody binding pair, wherein the first member of the binding pair is a coronavirus antigen, and wherein the sample comprises a plurality of IgG and IgM isotype antibodies that bind to the coronavirus antigen; directing an optical signal having the first wavelength to interact with the sensing surface over the second range of incident angles to generate a second SPR signal; generating a series of images of the second SPR signal over a first time interval using the detection component; determining a series of pixel positions that correspond to a minimum value of the second SPR signal over the first time interval; determining a rate of change of the series of pixel positions that corresponds to the minimum value of the second SPR signal over the first time interval; determining a plateau value of the second SPR signal based on the rate of change of the series of pixel positions that corresponds to the minimum value of the second SPR signal over the first time interval to generate a combined IgM IgG SPR test value; contacting the sensing surface with an IgG stripping agent; directing an optical signal having the first wavelength to interact with the sensing surface over the second range of incident angles to generate a third SPR signal; generating a series of images of the third SPR signal over a second time interval using the detection component; determining a series of pixel positions that correspond to a minimum value of the third SPR signal over the second time interval; determining a rate of change of the series of pixel positions that corresponds to the minimum value of the third SPR signal over the second time interval; determining a plateau value of the third SPR signal based on the rate of change of the series of pixel positions that corresponds to the minimum value of the third SPR signal over the second time interval to generate an IgM SPR test value; comparing the combined IgM IgG SPR test value, the IgM SPR test value, and the SPR reference value to determine the coronavirus exposure status of the patient.

### BRIEF DESCRIPTION OF THE DRAWINGS

FIG. 1 is a depiction of a virus showing various antigens that can be targeted by antibodies.

FIG. 2 is a graph showing analyte levels as a function of time. The first set of peaks depicts the concentration of viral antigen and viral RNA as a function of time. The second set of peaks depicts the concentration of IgM and IgG antibodies as a function of time.

FIG. 3 is an illustration that depicts a workflow for detecting IgM and IgG antibodies that bind to SARS-CoV-2 spike protein, and for detecting whether a patient has been recently infected, is late stage infected, has recovered, or has never been infected with SARS-CoV-2.

FIG. 4 is an illustration showing how the subject detection systems and methods can be used to quantitatively determine the concentration of IgG in a donor plasma sample for use in convalescent plasma therapy and/or prophylaxis.

FIG. 5, Panel A is an illustration demonstrating the Surface Plasmon Resonance (SPR) technique for analyzing a sample. Panel B is a graph showing relative response as a function of SPR angle.

FIG. 6 is an illustration of an example of an injection molded sensor. The sensor and the sensing surface are referenced.

FIG. 7 is an illustration of another example of an injection molded sensor.

FIG. 8 is an illustration of another example of an injection molded sensor. The depicted sensor is configured to direct a first optical signal to interact with a sensing surface at an incident angle of 42.04 degrees, and to direct a second optical signal to interact with the sensing surface at an incident angle of 64.44 degrees.

FIG. 9 is an illustration of another example of an injection molded sensor. The depicted sensor is configured to direct a first optical signal to interact with a sensing surface at an incident angle of 42.04 degrees, and to direct a second optical signal to interact with the sensing surface at an incident angle of 64.44 degrees.

FIG. 10, Panel A is a side view illustration of a sensor. Panel B is a bottom view illustration of a sensor.

FIG. 11 is a perspective illustration of a sensor.

FIG. 12, Panels A and B show side view illustrations of a sensor.

FIG. 13 is and end view illustration of a sensor.

FIG. 14 is a transparent rendering of a sensor.

FIG. 15, Panels A-E show images and graphs of SPR signals collected over different time intervals using the methods described herein.

### DETAILED DESCRIPTION

Sensors, systems and methods for detecting analytes in a sample are provided. Aspects of the subject methods include contacting a sensing surface of a sensor with a sample, and generating one or more data sets over a time interval, wherein the data sets are used to determine the presence or absence of a member of a binding pair in the sample. The subject methods find use in determining the presence or absence of one or more analytes in a sample, such as a biological sample (e.g., blood), and in the diagnosis and/or monitoring of various diseases and disorders, such as, e.g., infection with a virus.

Before the present invention is described in greater detail, it is to be understood that this invention is not limited to particular aspects described, as such may, of course, vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual aspects described and illustrated herein has discrete components and features which can be readily separated from or combined with the features of any of the other several aspects without departing from the scope or spirit of the present invention. Any recited method can be carried out in the order of events recited or in any other order which is logically possible.

### Definitions

The term “sensing surface” as used herein refers to a surface of a sensor that is configured to contact an external medium.

The terms “incident angle” or “angle of incidence” as used interchangeably herein refer to an angle that is formed between a beam of light that is directed toward a planar surface, and a line that is perpendicular to the same planar surface.

The term “facet” as used herein refers to a substantially planar portion of a surface (e.g., an interior surface or an exterior surface) of a sensor.

The term “semitransparent film” as used herein refers to a film that is partially transparent to light and facilitates surface plasmon/polariton generation.

The terms “reflective coating” and “reflective film”, as used interchangeably herein, refer to a coating or a film, respectively, that are capable of reflecting light or other radiation. The terms “semitransparent film” and “reflective film” or “reflective coating” as used herein are not mutually exclusive, and a given film can be both a semitransparent film as well as a reflective film.

The term “noble metal” as used herein refers to a metallic element that is resistant to corrosion in moist air. Non-limiting examples of noble metals include Copper (Cu), Ruthenium (Ru), Rhodium (Rh), Palladium (Pd), Silver (Ag), Rhenium (Re), Osmium (Os), Iridium (Ir), Platinum (Pt), Gold (Au), Mercury (Hg), or combinations thereof.

The term “adhesion layer” as used herein refers to a layer of material that is formed on a sensing surface or on a facet, and which facilitates adhesion of a coating material (e.g., a reflective film or a semitransparent film) to the sensing surface or facet.

The term “coated region” as used herein with reference to a sensing surface or facet means a region of the sensing surface or facet that is covered with a coating (e.g., a semitransparent film, a reflective coating, and/or an adhesion layer). The term “non-coated region” as used herein with reference to a sensing surface or facet means a region of the sensing surface or facet that is not covered with a coating.

The term “sensor” as used herein refers to a structure that comprises a sensing surface and that is configured to be removably coupled to an optical chassis. In certain embodiments, a sensor can comprise optical components, such as facets, that are configured to direct one or more optical signals to interact with the sensing surface over one or more predetermined ranges of incident angles.

The term “optical chassis” as used herein refers to a structure that supports and/or contains one or more optical components.

The term “optical signal” as used herein refers to a signal that comprises photons.

The term “critical angle” as used herein refers to an angle of incidence above which (e.g., at an angle of incidence having a larger angular value than the critical angle) total internal reflection occurs.

The term “pixel position” as used herein refers to the position of a pixel on a coordinate system, such as, e.g., an x,y coordinate plane.

The term “compare” as used herein with respect to comparing pixel positions refers to measuring a difference in position of two or more pixels on a coordinate plane. Comparing of pixel positions can be qualitative or quantitative.

The term “reference feature” as used herein refers to one or more data points that do not vary with time, or a component that is configured or adapted to generate one or more data points that do not vary with time.

The term “opto-mechanical reference” or “OMR” refers to a component that is configured or adapted to place a physical obstruction in the path of one or more optical signals and to thereby generate one or more reference signals that do not vary with time, and that can be detected and analyzed by a detection component.

The terms “delta pixel position” or “delta pixel value” as used herein refer to a numerical value that represents a difference in position between two pixels on a coordinate system.

The term “external environment parameter” as used herein refers to a characteristic of an environment that is external to a subject sensor or system. A non-limiting example of an external environment parameter is the temperature of a room in which a sensor is operated.

The term “corrected” as used herein with respect to a data value refers to a data value that has undergone a mathematical manipulation, e.g., has been multiplied or divided by a numerical value to correct or normalize the data value based on a given parameter (e.g., an external environment parameter, or a reference value).

The term “reference-corrected” as used herein with respect to a data value or a mathematical function (e.g., an SPR function) refers to a data value or mathematical function that has undergone a mathematical manipulation, e.g., has been multiplied or divided by at least one numerical value obtained from one or more reference features to correct or normalize the data value based on the at least one numerical value obtained from the reference feature.

The term “calibration data set” as used herein refers to a collection of one or more data points that represent a relationship between a measurement standard and a characteristic that is measured by a subject sensor and/or system.

The term “function” as used herein refers to a mathematical operation, or graphical representation thereof, wherein a unique y coordinate value is assigned to every x coordinate value.

The term “minimum value” as used herein refers to the lowest numerical value of a function in an image frame and on a given coordinate system.

The term “maximum value” as used herein refers to the highest numerical value of a function in an image frame and on a given coordinate system.

The term “derivative” as used herein refers to a rate of change of a function. The value of a derivative of a function is the slope of the tangent line at a point on a graph representing the function.

The term “plateau value” as used herein refers to a y-value of a function over a region where the function has a substantially constant, or steady-state, y-value.

The term “quality parameter” as used herein refers to an aspect of a subject sensor or system that is required for optimal functioning of the sensor or system.

The term “surface plasmon resonance” or “SPR” as used herein refers to a resonant oscillation of conduction electrons at an interface between a negative and a positive permittivity material that is stimulated by incident light.

The term “optical signal manipulation component” as used herein refers to a component that is capable of manipulating one or more features of an optical signal. An optical signal manipulation component can include any number of individual components, which individual components can act in parallel and/or in series to manipulate one or more characteristics of an optical signal. Non-limiting examples of optical signal manipulation components include: beam splitters, spatial filters, filters that reduce external ambient light, lenses, polarizers, and optical waveguides.

The term “removably couple” as used herein refers to connecting two or more components in such a way that the connection is reversible, and the components can be separated from one another.

The term “retention component” as used herein refers to a component that is configured to retain one or more components in a fixed position with respect to another component.

The term “alignment component” as used herein refers to a component that is configured to provide functional and/or structural alignment between two or more components that are operably coupled.

The term “kinematic mounting component” as used herein refers to a mounting component that provides a number of constraints that is equal to the number of degrees of freedom in the component being mounted.

The term “benchtop system” as used herein refers to a system that is configured to be disposed on a surface of, e.g., a laboratory benchtop, or another suitable substrate, during operation.

The term “hand-held system” as used herein refers to a system, or a component thereof, that is configured to be held in a user's hand during operation.

The terms “subject” or “patient” as used herein refer to any human or non-human animal.

### Sensors and Systems

Aspects of the invention include sensors and systems configured to carry out the subject methods, e.g., to detect the presence of an analyte in a sample. In certain embodiments, the subject systems include an optical sensor having at least one sensing surface and configured to direct a first optical signal to interact with the sensing surface at a first incident angle, and to direct a second optical signal to interact with the sensing surface at a second incident angle. In some embodiments, the subject systems further include an optical chassis that includes an optical signal generation component and a detection component. Each of these components is now further described in greater detail.

Aspects of the subject sensors and systems are described, for example, in published PCT applications WO2017053853, WO2017083580 and WO2016201189, the disclosures of which are incorporated by reference herein in their entireties.

### Sensors

As summarized above, aspects of the invention include sensors that include at least one sensing surface, and that are configured to direct a first optical signal to interact with the sensing surface at a first incident angle, and to direct a second optical signal to interact with the sensing surface at a second incident angle. By directing optical signals to interact with the sensing surface at two different incident angles, the subject sensors are capable of generating data from the sensing surface for two or more different media (e.g., air and water), and detecting the data using the same detection component. As such, data obtained from different media can be captured in the same field of view, or image frame, of a detection component, and can then be analyzed by the detection component. Analysis of the data can then be used to determine one or more characteristics of the media. The inclusion of data from the sensing surface for different media in the same field of view, or image frame, of the detection component provides an internal reference within the data that can be used in analysis (e.g., can be used for calibration of the sensor and/or for analyzing an unknown sample). As described further herein, in some embodiments, a sensor can include a reference feature that can be used in data analysis. In some embodiments, a sensor comprises a reference feature that creates a reference signal in an image frame of the detection component, and one or more pixel positions of the reference signal can be used as an internal reference for purposes of data analysis (e.g., can be used for calibration of the sensor and/or for analyzing a known or unknown sample).

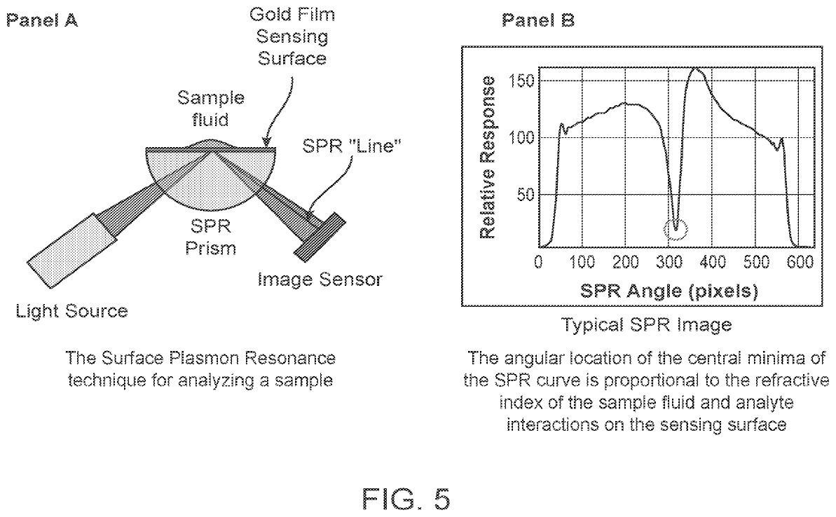

The subject sensors include at least one sensing surface that comprises a semitransparent film, wherein the semitransparent film comprises a noble metal. The semitransparent film facilitates surface plasmon resonance (SPR)-based analysis of a medium in contact with the sensing surface. SPR is a phenomenon that occurs when light is incident on a sensing surface at a particular angle, so that the reflected light is extinguished. At a particular angle of incident light, the intensity of the reflected light shows a characteristic curve of diminishing intensity, well defined by mathematical equations. The angle of incident light that corresponds to a reflectivity minimum of the curve is influenced by the characteristics of the semitransparent film and the external medium that is in contact therewith. FIG. 5, Panel A provides an illustrative overview of the SPR technique for analyzing a sample. FIG. 5, Panel B provides a graph of an SPR signal (i.e., an SPR signal curve, or function), demonstrating the relative minimum of the SPR curve, and indicating the position corresponding to a reflectivity minimum of the SPR signal curve. In some embodiments, aspects of the invention include determining a pixel position corresponding to a reflectivity minimum of an SPR signal curve represented on an image that is generated by a detection component (described further herein).

In some embodiments, the semitransparent film on the sensing surface can range in thickness from about 0.5 nm up to about 200 nm, such as about 1 nm, 5 nm, 10 nm, 15 nm, 20 nm, 25 nm, 30 nm, 35 nm, 40 nm, 41 nm, 42 nm, 43 nm, 44 nm, 45 nm, 46 nm, 47 nm, 48 nm, 49 nm, 50 nm, 51 nm, 52 nm, 53 nm, 54 nm, 55 nm, 60 nm, 65 nm, 70 nm, 75 nm, 80 nm, 85 nm, 90 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, or 195 nm. A semitransparent film can be deposited on a surface of a sensor using any suitable technique, for example, thin film deposition techniques (e.g., atomic layer deposition (ALD), chemical vapor deposition (CVD), evaporative deposition, metal organic chemical vapor deposition (MOCVD), sputtering, etc.), or any combination thereof. Non-limiting examples of noble metals that can be used in a semitransparent film in accordance with embodiments of the subject sensors include Copper (Cu), Ruthenium (Ru), Rhodium (Rh), Palladium (Pd), Silver (Ag), Rhenium (Re), Osmium (Os), Iridium (Jr), Platinum (Pt), Gold (Au), Mercury (Hg), or any combination thereof. In some embodiments, a semitransparent film on a sensing surface can be composed of a plurality of discrete layers of material, wherein the material in each layer can be selected from the noble metals described above, or any combination thereof (e.g., alloys thereof, such as alloys of 2, 3, 4, 5, 6, 7, or 8 or more different noble metals). In some embodiments, a sensing surface can comprise a substrate, such as, e.g., a microscope slide, having one side that is at least partially coated with a semitransparent film. In such embodiments, the substrate can be operably coupled to the sensor to provide a sensing surface.

In some embodiments, a sensor can include an adhesion layer that is deposited on a sensing surface between the sensor (or substrate) and a semitransparent film. An adhesion layer in accordance with embodiments of the invention serves to promote adhesion of the semitransparent film to the sensing surface, and can modulate one or more properties of an optical signal passing through the sensor. For example, in some embodiments, an adhesion layer can comprise a material that improves a desired property of an optical signal that passes through the adhesion layer. In some embodiments, the thickness and material composition of an adhesion layer are selected to favorably manipulate a property of an optical signal that passes through the adhesion layer. In some embodiments, a material having a desired refractive index (RI) is selected to modulate a characteristic of an optical signal that passes through the adhesion layer. In some embodiments, the adhesion layer comprises a material that modulates a characteristic of an optical signal passing therethrough, e.g., reduces the amount of noise in the optical signal.

In some embodiments, an adhesion layer can range in thickness from about 0.5 nm up to about 200 nm, such as about 1 nm, 1.5 nm, 2 nm, 2.5 nm, 3 nm, 3.5 nm, 4 nm, 4.5 nm, nm, 5.5 nm, 6 nm, 6.5 nm, 7 nm, 7.5 nm, 8 nm, 8.5 nm, 9 nm, 9.5 nm, 10 nm, 15 nm, 20 nm, nm, 30 nm, 35 nm, 40 nm, 45 nm, 50 nm, 55 nm, 60 nm, 65 nm, 70 nm, 75 nm, 80 nm, 85 nm, 90 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, or 195 nm. An adhesion layer can be deposited on a surface of the sensor using any suitable technique, for example, thin film deposition techniques (e.g., atomic layer deposition (ALD), chemical vapor deposition (CVD), evaporative deposition, metal organic chemical vapor deposition (MOCVD), sputtering, etc.), or any combination thereof. Non-limiting examples of materials that can be used in an adhesion layer in accordance with embodiments of the subject sensors include Chromium (Cr), TiO₂, TOₓ, SiO₂, SiOₓ, or any combination thereof (e.g., mixtures or alloys thereof).

Sensing surfaces in accordance with embodiments of the invention can have any suitable size and shape. In some embodiments, a sensing surface can be square, rectangular, trapezoidal, octagonal, elliptical, or circular in shape, or any combination thereof. The surface area of a sensing surface can vary, and in some embodiments can range from about 1 mm² up to about 10 mm², such as about 2 mm², 3 mm², 4 mm², 5 mm², 6 mm², 7 mm², 8 mm², or 9 mm².

In certain embodiments, a sensing surface can comprise a coated region and a non-coated region. In some embodiments, a coated region comprises a percentage of the area of the sensing surface that ranges from about 10% up to 100%, such as about 15%, 20%, 25%, 30%, 35%, 40%, 45%, 50%, 55%, 60%, 65%, 70%, 75%, 80%, 85%, 90%, or 95% of the area of the sensing surface. In certain embodiments, an entire sensing surface is coated with a semitransparent film.

A coated region in accordance with embodiments of the invention can have any suitable shape. In some embodiments, a coated region of a sensing surface can be square, rectangular, trapezoidal, octagonal, elliptical, or circular in shape, or any combination thereof. In some embodiments, a sensing surface can comprise a plurality of discrete coated regions, such as 2, 3, 4, 5, 6, 7, 8, 9 or 10 discrete coated regions. A coated region of a sensing surface can be located in any suitable position on a sensing surface. For example, in some embodiments, a coated region can be centered on a sensing surface, while in some embodiments, a coated region can be, e.g., located on one particular side of a sensing surface, located along one or more sides of a sensing surface, or the like. In some embodiments, approximately half of the sensing surface comprises a coated region, while approximately half of the sensing surface comprises a non-coated region. In some embodiments, approximately two thirds (approximately 66%) of the sensing surface comprises a coated region, while approximately one third (approximately 33%) of the sensing surface comprises a non-coated region. In certain embodiments, the entire surface of a sensing surface is a coated region (i.e., 100% of the sensing surface is coated with a semitransparent film).

In some embodiments, a non-coated region of a sensing surface facilitates analysis of a critical angle associated with the sensor. The critical angle is the incident angle above which total internal reflection occurs. The critical angle is influenced by the characteristics of the material from which the sensor is made, and is not influenced by the external medium that is in contact with a sensing surface of the sensor. As such, the critical angle for a given sensor can serve as an internal reference during analysis. In some embodiments, aspects of the invention include determining a critical angle for a sensor, as well as determining a pixel position corresponding to the critical angle on an image that is generated by a detection component (described further herein).

Sensors in accordance with embodiments of the invention can have any suitable size and shape. In some embodiments, a sensor has a hemi-cylinder shape, having a planar surface and a curved surface, wherein the sensing surface is disposed on the planar surface. In some embodiments, a sensor comprises a conical or frustoconical shape. In some embodiments, a sensor can have a concave shape, such that the sensor comprises an interior surface (e.g., a surface inside the concavity) and an exterior surface. In some embodiments, a sensor can have a frustoconical, concave shape.

In some embodiments, a sensor can have a length dimension that ranges from about 1 to about 20 cm, such as 2 cm, 3 cm, 4 cm, 5 cm, 8 cm, 10 cm, 12 cm, 14 cm, 16 cm, or 18 cm. In some embodiments, a sensor can have a width dimension that ranges from about 1 to about 20 cm, such as 2 cm, 3 cm, 4 cm, 5 cm, 8 cm, 10 cm, 12 cm, 14 cm, 16 cm, or 18 cm. In some embodiments, a sensor can have a height dimension that ranges from about 1 to about 20 cm, such as 2 cm, 3 cm, 4 cm, 5 cm, 8 cm, 10 cm, 12 cm, 14 cm, 16 cm, or 18 cm. In some embodiments, a sensor can have a diameter that ranges from about 1 to about 20 cm, such as 2 cm, 3 cm, 4 cm, 5 cm, 8 cm, 10 cm, 12 cm, 14 cm, 16 cm, or 18 cm.

In some embodiments, a sensor can comprise one or more facets that are configured to direct an optical signal in a given direction (e.g., to reflect off the facet at a given angle). Facets in accordance with embodiments of the invention can have any suitable area, and in some embodiments can range in area from about 1 mm² up to about 100 mm², such as about 5 mm², 10 mm², 15 mm², 20 mm², 25 mm², 30 mm², 35 mm², 40 mm², 45 mm², 50 mm², 55 mm², 60 mm², 65 mm², 70 mm², 75 mm², 80 mm², 85 mm², 90 mm², or 95 mm². Facets in accordance with embodiments of the sensor can have any suitable shape, and in some embodiments can be square, rectangular, trapezoidal, octagonal, elliptical, or circular in shape, or any combination thereof.

Sensors in accordance with embodiments of the invention can have any suitable number of facets on a given surface of the sensor. For example, in some embodiments, a sensor can have a number of facets ranging from 1 up to 10, such as 2, 3, 4, 5, 6, 7, 8 or 9 facets on a given surface of the sensor. In certain embodiments, a sensor can have one or more facets on an internal surface, such as 1, 2, 3, 4, 5, 6, 7, 8, 9, or 10 facets on an internal surface, and can also have one or more facets on an external surface, such as 1, 2, 3, 4, 5, 6, 7, 8, 9, or 10 facets on an external surface. In some embodiments, a facet can be coated with an optically reflective material to enhance the ability of the facet to reflect an optical signal. In some embodiments, a plurality of facets can have a different shape and/or area. In some embodiments, a plurality of facets can have the same shape and/or area.

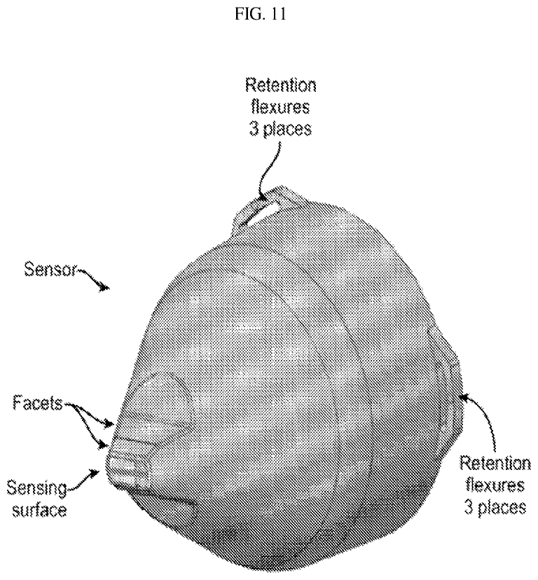

In certain embodiments, one or more facets can be coated with a reflective coating (e.g., a reflective film, or an optically reflective material). In some embodiments, all of the facets of a sensor can be coated with a reflective coating. In some embodiments, certain facets on a sensor are coated with a reflective coating, whereas other facets on the same sensor are not coated with a reflective coating. In some embodiments, the entire surface of a selected facet can be coated with a reflective coating. In some embodiments, only a portion or section of the surface of a particular facet is coated with a reflective coating. In a preferred embodiment, a plurality of “shoulder” facets are coated with a reflective gold coating. For example, in one preferred embodiment, the facets that are labeled in FIG. 11 (as well as those that are symmetrically located on the opposite side of the sensing surface) are coated with a reflective coating (e.g., a reflective gold coating).

In some embodiments, a reflective coating on the surface of a facet can range in thickness from about 0.1 nm up to about 1,000 nm (1 μm), such as about 0.5 nm, about 1 nm, about 5 nm, about 10 nm, about 20 nm, about 30 nm, about 40 nm, about 50 nm, about 60 nm, about 70 nm, about 80 nm, about 90 nm, about 100 nm, about 150 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, or about 950 nm or more. A reflective coating can be deposited on a surface of a facet using any suitable technique, such as, for example, thin film deposition techniques (e.g., atomic layer deposition (ALD), chemical vapor deposition (CVD), evaporative deposition, metal organic chemical vapor deposition (MOCVD), sputtering, etc.), or any combination thereof. Non-limiting examples of noble metals that can be used in a reflective film in accordance with embodiments of the subject sensors include Copper (Cu), Ruthenium (Ru), Rhodium (Rh), Palladium (Pd), Silver (Ag), Rhenium (Re), Osmium (Os), Iridium (Jr), Platinum (Pt), Gold (Au), Mercury (Hg), or any combination thereof. In a preferred embodiment, a reflective coating comprises gold (Au).

In some embodiments, a sensor can include an adhesion layer that is deposited on one or more facets and is positioned between the sensor (or substrate) and a reflective coating on the facet. An adhesion layer in accordance with embodiments of the invention serves to promote adhesion of the reflective coating to the facet, and can modulate one or more properties of an optical signal that is reflected off the facet. For example, in some embodiments, an adhesion layer can comprise a material that improves a desired property of an optical signal that is reflected off a particular facet. In some embodiments, the thickness and material composition of an adhesion layer are selected to favorably manipulate a property of an optical signal that is reflected off a particular facet.

In some embodiments, an adhesion layer can range in thickness from about 0.5 nm up to about 200 nm, such as about 1 nm, 1.5 nm, 2 nm, 2.5 nm, 3 nm, 3.5 nm, 4 nm, 4.5 nm, 5 nm, 5.5 nm, 6 nm, 6.5 nm, 7 nm, 7.5 nm, 8 nm, 8.5 nm, 9 nm, 9.5 nm, 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, 85 nm, 90 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, or 195 nm. An adhesion layer can be deposited on a surface of the sensor (e.g., on a facet of the sensor) using any suitable technique, for example, thin film deposition techniques (e.g., atomic layer deposition (ALD), chemical vapor deposition (CVD), evaporative deposition, metal organic chemical vapor deposition (MOCVD), sputtering, etc.), or any combination thereof. Non-limiting examples of materials that can be used in an adhesion layer in accordance with embodiments of the subject sensors include Chromium (Cr), TiO₂, TOₓ, SiO₂, SiOₓ, or any combination thereof (e.g., mixtures or alloys thereof).

In some embodiments, a sensor includes a first member of a binding pair immobilized on at least a portion of the sensing surface (e.g., on a coated region of a sensing surface). In use, the subject sensors are contacted with a sample that contains the second member of the binding pair, and the binding activity between the two members of the binding pair can be used to determine the presence and/or concentration of the second member of the binding pair in the sample.

Any of a variety of suitable binding pairs can be utilized in connection with the subject sensors, systems, and methods described herein, including, for example: a receptor/ligand pair, an antigen/antibody pair, a lectin/carbohydrate pair, an enzyme/substrate pair, a biotin/avidin pair, a biotin/streptavidin pair, a DNA or RNA aptamer binding pair, a peptide/aptamer binding pair, a metal/metal binding peptide pair, or any combination thereof. In one preferred embodiment, the binding pair is an antigen/antibody binding pair. In one preferred embodiment, the binding pair is an antigen/antibody binding pair, wherein a plurality of different antibody isotypes (e.g., IgG, IgM, IgA, IgE, and IgD, or any combination thereof) are present in a test sample, and the relative concentration of one or more of such antibody isotypes is determined using the methods described herein.

In one preferred embodiment, an antigen is a viral protein antigen, such as, for example, a viral membrane protein, a viral envelop protein, or a viral nucleoprotein. In one preferred embodiment, a viral protein antigen comprises a coronavirus spike protein. In one preferred embodiment, the coronavirus spike protein is a SARS-CoV-2 spike protein. In one preferred embodiment, the SARS-CoV-2 spike protein is an S1 or an S2 subunit protein.

In some embodiments, a sensor can include one or more identification components that are configured to communicate identifying information to another component of a system (e.g., to a component of an optical chassis, to a processor, etc.). For example, in some embodiments, a sensor can include an identification component that provides an optical chassis with information regarding, e.g., a type of semitransparent film disposed on the sensing surface of the sensor, a configuration of coated and non-coated regions on a sensing surface of the sensor, a configuration of facets in the sensor, etc. In some embodiments, a system is configured to respond to identifying information communicated by a sensor. For example, in certain embodiments, a system can be configured to receive identifying information from a sensor, and in response, configure the system to carry out a particular method of analysis (e.g., configure the system to generate one or more optical signals having a particular wavelength or wavelengths). Identification components in accordance with embodiments of the invention can have any suitable structure, and can include, for example, bar codes, magnetic strips, computer-readable chips, and the like. Systems in accordance with embodiments of the invention can be configured with a corresponding identification component that is configured to receive and/or identify identification information from an identification component on a sensor.

Aspects of the subject sensors include retention components that are configured to retain a sensor in a fixed position with respect to another component of a subject system (e.g., an optical chassis, described further herein). Retention components in accordance with embodiments of the invention can have any suitable shape and dimensions, and can take the form of, e.g., tabs or flanges that extend from one or more portions of a subject sensor. In some embodiments, a sensor can include a retention component that is configured to removably couple the sensor to another component, such as, e.g., an optical chassis. In some embodiments, a sensor is configured to be removably coupled and/or de-coupled to an optical chassis in a touchless, or aseptic manner, meaning that an operator can accomplish the coupling of the sensor to the optical chassis without compromising the sterility of the sensor, and can accomplish de-coupling the sensor from the optical chassis without having to physically contact the sensor.

Aspects of the subject systems include one or more sensor mounting components that are configured to facilitate aseptic handling of a sensor, as well as coupling (e.g., removable coupling) of the sensor to an optical chassis. For example, in certain embodiments a sensor mounting component is configured to hold a sensor in an aseptic manner, allow a user to couple the sensor to an optical chassis, and then disengage from the sensor, leaving the sensor coupled to the optical chassis in an aseptic manner. Sensor mounting components in accordance with embodiments of the invention can have any suitable dimensions, and in some embodiments include a surface that is complementary to at least a portion of a sensor. In some embodiments, a sensor mounting component is configured to cover at least a portion of an external surface of a sensor so that the covered portion of the sensor is not accessible to an external environment until the sensor mounting component is disengaged from the sensor. In some embodiments, a sensor mounting component is adapted for sterilization via any suitable technique, and is adapted to maintain its functionality after the sterilization has been completed. Sterilization techniques are well known in the art and include, e.g., heat sterilization, gamma irradiation, chemical sterilization (e.g., ethylene oxide gas sterilization), and many others. Aspects of the invention include sensor mounting components that are adapted for sterilization without altering their functionality in any appreciable manner. In some embodiments, a sensor mounting component is configured to allow sterilization of a sensor while the sensor and the sensor mounting component are coupled to one another.

Aspects of the subject sensors include one or more kinematic mounting components that are configured to provide a number of constraints that is equal to the number of degrees of freedom of the component being mounted. For example, for a three dimensional object having six degrees of freedom, kinematic mounting components that provide six constraints can be used to mount a sensor on an optical chassis (described further below).

Aspects of the subject sensors include one or more alignment components that are configured to align the sensor with one or more components of an optical chassis (described further below). In some embodiments, an alignment component can comprise a tapered centering component that is configured to align a sensor with an optical chassis.

The subject sensors can be made from any of a variety of suitable materials, including but not limited to glass, optical grade plastics, polymers, combinations thereof, and the like. Non-limiting examples of suitable materials include polymethylmethacrylate (PMMA), polycarbonate (PC), polystyrene (PS), cyclo-olefin polymers (e.g., ZEONEX® E48R), sapphire, diamond, quartz, zircon (zirconium), and the like, or any combination thereof. In some embodiments, a material that is used to make a subject sensor can have a refractive index that ranges from about 1.2 up to about 2.0, such as 1.21, 1.22, 1.23, 1.24, 1.25, 1.26, 1.27, 1.28, 1.29, 1.3, 1.31, 1.32, 1.33, 1.34, 1.35, 1.36, 1.37, 1.38, 1.39, 1.4, 1.41, 1.42, 1.43, 1.44, 1.45, 1.46, 1.47, 1.48, 1.49, 1.5, 1.51, 1.52, 1.53, 1.54, 1.55, 1.56, 1.57, 1.58, 1.59, 1.6, 1.61, 1.62, 1.63, 1.64, 1.65, 1.66, 1.67, 1.68, 1.69, 1.7, 1.71, 1.72, 1.73, 1.74, 1.75, 1.76, 1.77, 1.78, 1.79, 1.8, 1.81, 1.82, 1.83, 1.84, 1.85, 1.86, 1.87, 1.88, 1.89, 1.9, 1.91, 1.92, 1.93, 1.94, 1.95, 1.96, 1.97, 1.98, or 1.99. Those of skill in the art will recognize that any material having suitable optical properties can be used in the subject sensors. Sensors in accordance with embodiments of the invention can be fabricated using any suitable technique, such as machining, 3D-printing, and/or molding (e.g., injection molding). In some embodiments, a sensor can be fabricated using a suitable technique, and can then be further processed to deposit one or more compositions on a surface of the sensor (e.g., a semitransparent film, adhesion layer, or a reflective coating). In some embodiments, a sensor is disposable, and can be discarded after one or more uses. In some embodiments, a sensor is adapted for repeated use, for example, is adapted to be cleaned and sterilized following use, and then used again.

As reviewed above, aspects of the invention include sensors that are configured to direct a first optical signal to interact with a sensing surface at a first incident angle, and to direct a second optical signal to interact with the sensing surface at a second incident angle so that data from the sensing surface for two different test media (e.g., air and a biological sample, e.g., a tear film) can be captured in the same field of view, or image frame, of a detection component. In some embodiments, a sensor is configured to direct a first optical signal to interact with a sensing surface over a narrow range of first incident angles, and to direct a second optical signal to interact with the sensing surface over a narrow range of second incident angles in order to generate data in the same field of view, or image frame, of a detection component, as reviewed above. In some embodiments, a narrow range of incident angles spans a number of degrees ranging from about 2 to about 10 degrees, such as about 3, 4, 5, 6, 7, 8 or 9 degrees.

Without being held to theory, a range of first and second incident angles that are chosen for a sensor depends on the optical properties of the material that is used to fabricate the sensor, as well as the external medium to be analyzed by the sensor. As such, a first and second incident angle, or a first and second narrow range of incident angles, can differ for sensors that are composed of different materials, and a range of incident angles for a given sensor can be based on the anticipated refractive index of a sample being analyzed (e.g., a biological sample). In some embodiments, a sensor is configured to have a dynamic range of incident angles of clinical significance, wherein the sensor is configured to direct one or more optical signals to interact with a sensing surface over a range of incident angles that facilitate analysis of a sample and provide data having clinical significance (e.g., data that facilitate the determination of the osmolarity of a biological sample, e.g., a tear film). Those of skill in the art will appreciate that different first and second incident angles, or ranges thereof, can be selected based on, e.g., the optical properties of the material that is used to fabricate the sensor, the properties of the external media that will be brought into contact with the sensing surface (e.g., a biological sample and/or a reference medium), the properties of the semitransparent film, and/or the properties of the adhesion layer (if present), in order to generate data in the same field of view of a detection component from the sensing surface for different test media, or different combinations of reference and test media (e.g., air and water, air and tear fluid, etc.). In some embodiments, a range of incident angles broadly spans about 35 degrees to about 75 degrees, such as about 36, 37, 38, 39, 40, 41, 42, 43, 44, 45, 46, 47, 48, 49, 50, 51, 52, 53, 54, 55, 56, 57, 57, 59, 60, 61, 62, 63, 64, 65, 66, 67, 68, 69, 70, 71, 72, 73 or 74 degrees.

In some embodiments, a sensor, when coupled with an optical chassis (as described below) can be formed into a benchtop system that is configured for use in a laboratory setting, e.g., in a clinical laboratory setting. In some embodiments, a sensor, when coupled with an optical chassis (as described below) can be formed into a hand-held system. In a preferred embodiment, a hand-held system has dimensions that are similar to those of a pen. In use, a hand-held system can be held by, e.g., a physician, and contacted with a sample undergoing analysis.

In some embodiments, a sensor is adapted for sterilization via any suitable technique, and is adapted to maintain its functionality after the sterilization has been completed. Sterilization techniques are well known in the art and include, e.g., heat sterilization, gamma irradiation, chemical sterilization (e.g., ethylene oxide gas sterilization), and many others. Aspects of the invention include sensors that are adapted for sterilization without altering their functionality in any appreciable manner.

Aspects of the invention include kits that contain a plurality of sensors. In some embodiments, a kit can contain a plurality of identical sensors. In some embodiments, a kit can contain two or more sensors having different characteristics (e.g., a plurality of a first type of sensor, and a plurality of a second type of sensor). Kits in accordance with embodiments of the invention can comprise any suitable packaging, for example, can comprise airtight packaging (e.g., hermetically sealed packaging), vacuum sealed packaging, and the like. In certain embodiments, a kit can be sterile (e.g., the contents of the kit are sterile, and the kit packaging is configured to maintain the sterility of the contents). In some embodiments, a kit can comprise a plurality of sensors, wherein each individual sensor is separately sealed in sterile packaging. In some embodiments, a kit is not sterile, but is adapted for sterilization so that the kit can be sterilized at a point of use, e.g., at a clinician's office or at a hospital. In some embodiments, a kit can further include one or more sensor mounting components, as described herein.

In some embodiments, a sensor is storage stable and can be stored for an extended period of time, such as one to two years or more, while maintaining its functionality. In certain embodiments, a sensor can be provided in a kit with suitable packaging so that the sensor remains storage stable for an extended period of time. For example, in some embodiments, a sensor can be provided in airtight packaging or vacuum sealed packaging to facilitate storage stability for an extended period of time.

In one preferred embodiment, a sensor is fabricated from a cyclo-olefin polymer and has a frustoconical, concave shape, having an interior surface and an exterior surface, wherein the sensor comprises two facets on the interior surface and four facets on the exterior surface, as well as a sensing surface located on the exterior surface, and wherein the facets are configured to direct a first optical signal to interact with the sensing surface at an incident angle of about 42 degrees, and to direct a second optical signal to interact with the sensing surface at an incident angle of about 64 degrees. In this preferred embodiment, data from both air and water, or from both air and tear fluid, can be collected in the same field of view, or image frame, of a detection component, thereby providing an internal reference within the image that can be used in analysis.

In another preferred embodiment, a sensor is fabricated from a cyclo-olefin polymer and has a frustoconical, concave shape, having an interior surface and an exterior surface, wherein the sensor comprises two facets on the interior surface and four facets on the exterior surface, as well as a sensing surface located on the exterior surface of the sensor, and wherein the facets are configured to direct a first optical signal to interact with a sensing surface over a narrow range of incident angles that ranges from about 40 to about 45 degrees, and is configured to direct a second optical signal to interact with the sensing surface over a narrow range of incident angles that ranges from about 62 to about 67 degrees.

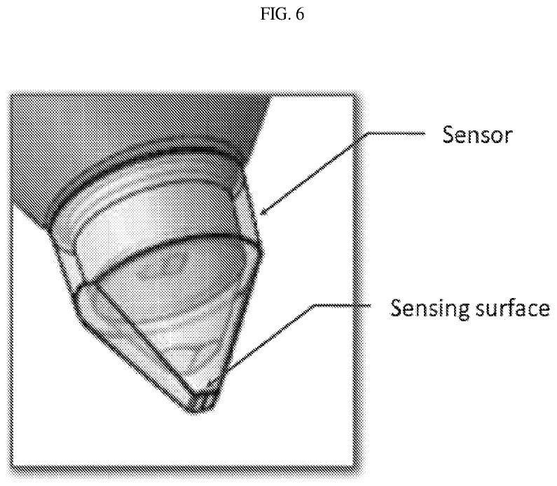

Turning now to FIG. 6, an illustration of a sensor in accordance with one embodiment of the invention is provided. The depicted embodiment is an injection molded clear plastic sensor with a sensing surface that comprises a gold film.

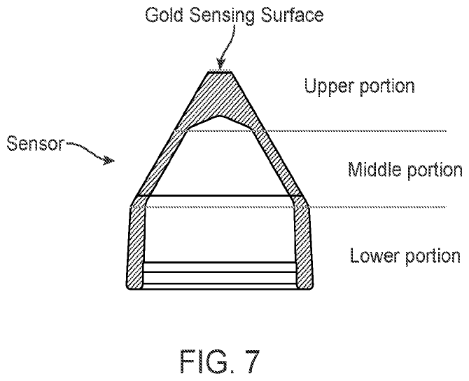

FIG. 7 is an illustration of another sensor in accordance with embodiments of the invention. In the depicted embodiment, the sensor comprises a sensing surface with a gold film. An upper portion of the depicted sensor functions as an SPR prism. A middle portion of the depicted sensor is a skirt portion, and the lower portion of the depicted sensor is a base portion that connects to an optical chassis (described further herein).

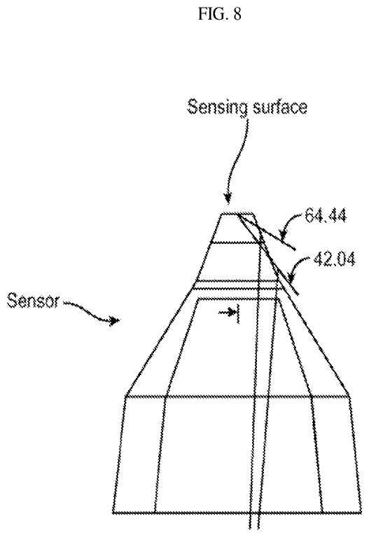

FIG. 8 is another illustration of a sensor in accordance with embodiments of the invention. In the depicted embodiment, the sensor is configured to direct a first optical signal to interact with the sensing surface at an incident angle of about 42.04 degrees, and is configured to direct a second optical signal to interact with the sensing surface at an incident angle of about 64.44 degrees.

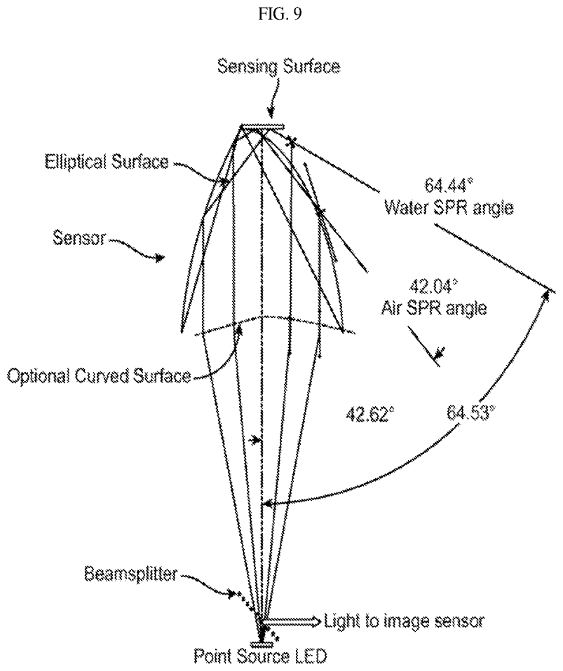

FIG. 9 is another illustration of a sensor in accordance with embodiments of the invention. In the depicted embodiment, the sensor is configured to direct a first optical signal to interact with the sensing surface at an incident angle of about 42.04 degrees, and is configured to direct a second optical signal to interact with the sensing surface at an incident angle of about 64.44 degrees. Further indicated are: a gold coating on the sensing surface, an elliptical outer surface of the sensor, an optional curved lower surface of the sensor, a point source LED and a beam splitter.

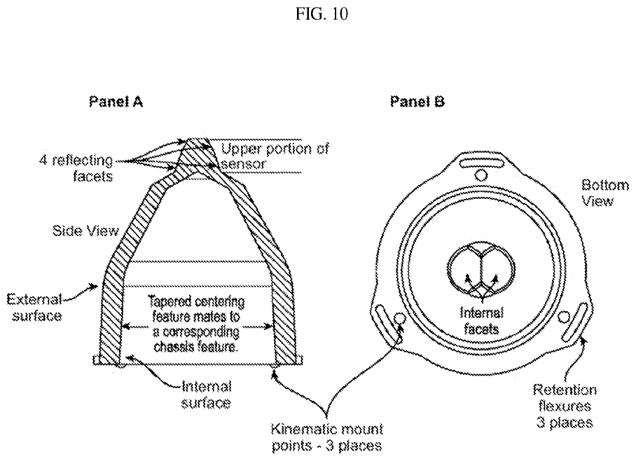

FIG. 10, Panel A is a side view of a sensor in accordance with embodiments of the invention having a frustoconical, concave shape with an internal surface and an external surface. In the depicted embodiment, an outer surface of the sensor has 4 reflecting facets and a tapered centering component that mates to an optical chassis. Panel B is a bottom view of the sensor, showing 2 facets on the internal surface of the sensor. Also depicted are retention components and kinematic mounting components.

FIG. 11 is a perspective view of the sensor depicted in FIG. 10. A plurality of retention fixtures is visible, as well as the sensing surface and 4 reflecting facets on the external surface of the sensor.

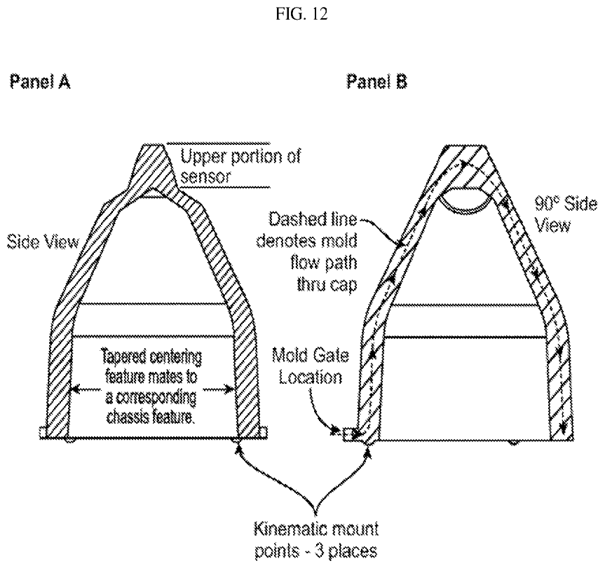

FIG. 12, Panel A is a side view of a sensor in accordance with embodiments of the invention having a frustoconical, concave shape with an internal surface and an external surface. In the depicted embodiment, an outer surface of the sensor has 4 reflecting facets and a tapered centering component that mates to an optical chassis. Panel B is side view of a sensor, showing a dashed line that indicates the flow of material through a mold during the process of fabricating the sensor. Also depicted are kinematic mounting locations.

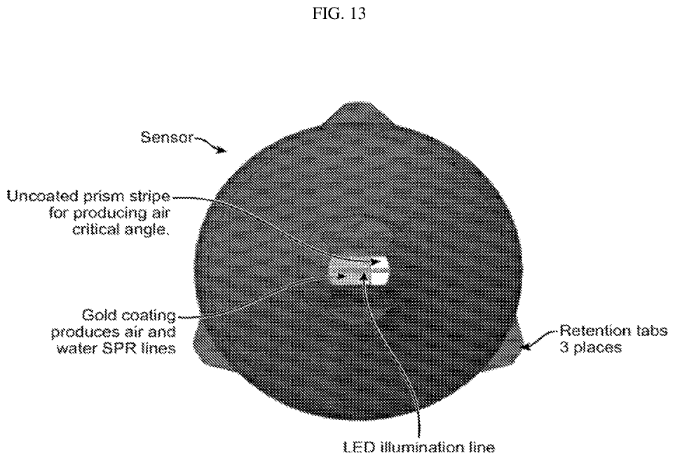

FIG. 13 is a top, end view of a sensor in accordance with embodiments of the invention. The depicted sensor includes a sensing surface that comprises coated and non-coated regions. Also depicted are three retention components, or tabs, that are configured to removably couple the sensor to an optical chassis.

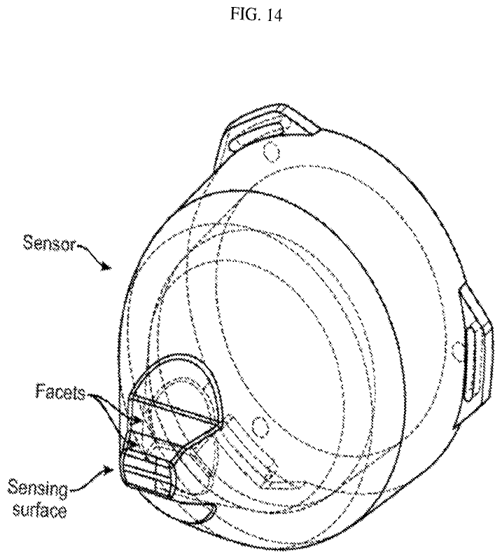

FIG. 14 is a transparent, perspective view of a sensor in accordance with embodiments of the invention.

### Optical Chassis

As summarized above, aspects of the invention include an optical chassis that comprises an optical signal generating component and a detection component. In some embodiments, an optical chassis can comprise an optical signal manipulation component. Optical chassis in accordance with embodiments of the invention are described, for example, in published PCT applications WO2017053853, WO2017083580 and WO2016201189, the disclosures of which are incorporated by reference herein in their entireties.

### Methods of Use

Aspects of the invention include methods of analyzing a sample using the subject sensors and systems to determine, e.g., the presence of an analyte, e.g., the presence of a member of a binding pair, in a sample. The subject methods involve contacting a sensing surface of a sensor with a medium to be tested (e.g., a reference medium, or a test sample having an unknown concentration of an analyte) for a sufficient period of time to carry out one or more of the subject methods. In some embodiments, a subject method can be carried out in a time period that is about 90 seconds or less, such as 80 seconds, 70 seconds, 60 seconds, 50 seconds, 40 seconds, 30 seconds, 20 seconds, 10 seconds, 5 seconds, 4 seconds, 3 second, 2 second, or 1 second or less, such as 0.5 seconds, 0.4 seconds, 0.3 seconds, 0.2 seconds, or 0.1 seconds or less, such as about seconds, 0.08 seconds, 0.07 seconds, 0.06 seconds, 0.05 seconds, 0.04 seconds, 0.03 seconds, 0.02 seconds, or 0.01 seconds or less, such as about 0.009 seconds, 0.008 seconds, seconds, 0.006 seconds, 0.005 seconds, 0.004 seconds, 0.003 seconds, 0.002 seconds, or seconds or less.

In some embodiments, the subject methods involve determining the presence of an analyte in a biological sample obtained from a patient or subject. The information can be used to assist a care giver or end user in diagnosing the patient or subject with a condition or disorder (e.g., infection with a virus) based on the results of the analysis. For example, in some embodiments, if a sample from a patient is determined to contain antibodies that bind to a viral antigen, then the care giver or end user can diagnose the patient as having been exposed to the virus.

The subject methods can be used to determine the presence of an analyte of interest in any suitable biological sample. Biological samples that can be analyzed using the subject methods include, without limitation: blood, plasma, serum, sputum, mucus, saliva, urine, feces, gastric and digestive fluid, tears, nasal lavage, semen, vaginal fluid, lymphatic fluid, interstitial fluids derived from tumorous tissue, ascites, cerebrospinal fluid, sweat, breast milk, synovial fluid, peritoneal fluid, amniotic fluid, or any combination thereof.

Any suitable volume of sample can be used in conjunction with the subject methods. In some embodiments, the volume of a sample ranges from about 5 nanoliters (nL) up to about 1 milliliter (mL), such as about 25, 50, 75, or 100 nL, such as about 200, 300, 400, 500, 600, 700, 800, 900 or 1,000 nL, such as about 5, 25, 50, 75 or 100 microliters (IL), such as about 200, 300, 400, 500, 600, 700, 800, 900 or 1,000 μL. In some embodiments, a sensing surface of a sensor is contacted directly to a sample, e.g., is placed in direct contact with the sample. In some embodiments, a sensing surface of a sensor is contacted directly to a biological sample without having to physically separate the sample from the patient. For example, in some embodiments, a sensing surface is contacted directly to a tear fluid of a patient while the tear fluid remains in or on the patient's eye. In some embodiments, a sensing surface is contacted directly to a patient's blood (e.g., in an open wound, or from a pool of blood produced from a finger stick) without physically separating the blood from the patient. In some embodiments, a sensing surface is contacted directly to a patient's saliva without physically removing the saliva from the patient's mouth. In some embodiments, a sample is separated from a patient prior to testing (e.g., a sample of blood is collected in a test tube, and blood from the test tube is subsequently removed and contacted with the sensor to carry out the analysis).

Aspects of the methods involve contacting a sensing surface of a sensor with a sample (e.g., a biological sample) and directing an optical signal having a first wavelength to interact with the sensing surface at a first incident angle and over a first time interval to generate a signal (e.g., an SPR signal) in response. In some embodiments, the methods involve directing a second optical signal having a second wavelength to interact with the sensing surface at the first incident angle over a second time interval while the sensing surface is in contact with a sample. In some embodiments, the first and second time intervals are the same. In some embodiments, the first and second time intervals are different. In some embodiments, the first and second optical signals are directed to interact with the sensing surface concurrently, whereas in some embodiments, the first and second optical signals are directed to interact with the sensing surface in a gated manner.

Aspects of the methods further involve generating a series of images of the SPR signals over the time intervals, and determining a series of pixel positions that correspond to a minimum value of the SPR signals over the time intervals. In some embodiments, the pixel positions that correspond to the minimum value of the SPR signals over the time intervals are used to generate a mathematical function that plots the pixel position of the minimum value of the SPR signals versus time, referred to herein as an SPR function. In some embodiments, the methods involve comparing the SPR function to the pixel position of at least one reference feature to generate a reference-corrected SPR function. In certain embodiments, the methods involve comparing one or more characteristics of a first SPR function, which is generated from a first optical signal having a first wavelength, to one or more characteristics of a second SPR function, which is generated from a second optical signal having a second wavelength. In some embodiments, the characteristic of the function is a derivative of the function. In some embodiments, the characteristic of the function is a plateau value of the function.

Aspects of the methods involve contacting a sensing surface of a sensor with a reference medium and directing an optical signal having a first wavelength to interact with the sensing surface at a second incident angle to generate a signal (e.g., an SPR signal or a critical angle signal) in response. In some embodiments, the methods involve directing one or more optical signals having different wavelengths to interact with the sensing surface at the second incident angle while the sensing surface is in contact with the reference medium.

Aspects of the methods involve measuring critical angle signals as well as SPR signals that are generated from a sensing surface while the sensing surface is in contact with a reference medium. In some embodiments, an SPR signal is generated by directing an optical signal to interact with a coated region of a sensing surface. In some embodiments, a critical angle signal is generated by directing an optical signal to interact with a non-coated region of a sensing surface. In some embodiments, the methods involve directing first and second optical signals having different wavelengths to interact with a coated region of a sensing surface to generate first and second SPR signals. In some embodiments, the methods involve directing first and second optical signals having different wavelengths to interact with a non-coated region of a sensing surface to generate first and second critical angle signals.

In some embodiments, the methods involve first contacting a sensing surface of a sensor with a reference medium or reference fluid (e.g., air, sterile water, a calibration solution having a known concentration of an analyte, etc.) and determining an SPR reference value, as described above, and then contacting the sensing surface with a test sample (e.g., a biological sample, e.g., blood), and determining an SPR test value, as described above, and then comparing the SPR reference value to the SPR test value to determine the presence of an analyte (e.g., a member of a binding pair) in the test sample using one or data analysis procedures as described herein.

In some embodiments, the methods involve directing an optical signal to interact with a sensing surface at one or more incident angles. For example, in some embodiments, the methods involve directing a first optical signal to interact with a sensing surface at a first incident angle, and directing a second optical signal to interact with a sensing surface at a second incident angle. In some embodiments, the methods involve directing one or more optical signals to interact with a sensing surface at a different incident angle, depending on the type of medium that is in contact with the sensing surface. For example, in some embodiments, the methods involve contacting a sensing surface with a test sample (e.g., a biological sample) and directing one or more optical signals to interact with the sensing surface at a first incident angle, and contacting the sensing surface with a second medium (e.g., a reference medium) and directing one or more optical signals to interact with the sensing surface at a second incident angle. In some embodiments, the methods involve first contacting the sensing surface with a reference medium (e.g., air, or a reference solution) to calibrate the sensor, verify one or more quality parameters of the sensor, and/or to obtain one or more reference values from the reference medium, and then contacting the sensing surface with a test sample (e.g., a biological sample, e.g., a blood sample) and determining the presence of an analyte (e.g., a member of a binding pair) in the test sample.

In certain embodiments, the methods involve directing optical signals of different wavelengths to interact with a sensing surface. As reviewed herein, the subject systems are configured to generate optical signals having any wavelength ranging from about 300 to about 1,500 nm. In some embodiments, the methods involve generating a first optical signal having a wavelength of about 855 nm, and generating a second optical signal having a wavelength of about 950 nm. In some embodiments, a plurality of optical signals can be directed to interact with a sensing surface simultaneously. For example, in some embodiments, two or more optical signals having different wavelengths are directed to interact with a sensing surface simultaneously. In some embodiments, a plurality of optical signals can be directed to interact with a sensing surface in a gated manner.

Aspects of the methods involve measuring changes in the intensity of one or more optical signals that are reflected from the sensing surface as a function of time while a test sample (e.g., a biological sample) is in contact with the sensing surface. Without being held to theory, the inventors have determined that as a member of a binding pair in the sample (e.g., an antibody) interacts with the other member of the binding pair immobilized on the sensing surface (e.g., an antigen to which the antibody binds), the refractive index close to the sensing surface changes, altering the angle of the minimum reflected light intensity, or SPR angle. The change in the SPR angle, and/or the rate of change of the SPR angle, is proportional to the concentration of the member of the binding pair in the sample. The position of the minimum reflected light intensity, or minimum value of the SPR signal, can therefore be measured as a function of time, and the resulting data can be analyzed to determine one or more characteristics of the sample, such as the concentration of the member of the binding pair in the sample, by comparison to a calibration data set.

Aspects of the methods involve signal processing of one or more signals that are received from a sensing surface (e.g., one or more SPR signals and/or critical angle signals). In some embodiments, a system includes signal processing capabilities that are configured to process a signal prior to analysis. For example, in some embodiments, the methods involve processing a signal to reduce noise prior to analysis. In some embodiments, the methods involve applying a Gaussian blur algorithm to a signal to reduce the amount of noise in the signal. In some embodiments, the methods involve applying low pass filtering to a signal to reduce the amount of noise in the signal.

Aspects of the methods involve detecting a signal using a detection component. In some embodiments, a detection component is configured to generate one or more images that are based on a signal received from a sensing surface. In some embodiments, a detection component is configured to generate a plurality of images from one or more signals that are received by an imaging component. For example, in some embodiments, a detection component is configured to generate a plurality of images per second once a sample (e.g., a reference medium or a test medium) has been placed in contact with a sensing surface of a sensor. In some embodiments, a detection component is configured to generate a plurality of images per second, such as 10, 20, 30, 40, 50, 60, 70, 80, 90, or a 100 or more images per second. In some embodiments, a detection component is configured to generate a video of one or more optical signals that are received from a sensor. In some embodiments, a detection component is configured to capture one or more image frames of a video, and to subject the one or more image frames to further processing, as described further below.

In some embodiments, a detection component has a field of view, and an image can be generated from a region of interest (ROI) within the field of view. In certain embodiments, the methods involve capturing data from a plurality of signals from a sensing surface in a single image frame. Capturing data from a plurality of signals in a single image frame provides an internal reference that can be used in the analysis of a sample.

Aspects of the methods involve data processing of an image that is generated from a detection component. In some embodiments, data processing involves applying a coordinate system (e.g., an x,y coordinate system) to an image. In some embodiments, each pixel, or a portion thereof, within a generated image can be assigned a specific x,y coordinate value. In some embodiments, each pixel within an image can be assigned a numerical value related to the intensity or color of light in the pixel. For example, in some embodiments, each pixel in an image is assigned a gray-scale value. In some embodiments, each pixel in an image is assigned a color value. In some embodiments, data processing involves performing a mathematical operation on a plurality of pixels. For example, in some embodiments, data processing involves calculating an average gray-scale value of a plurality of pixels. In some embodiments, data processing involves calculating an average gray-scale value of a column of pixels at a particular x coordinate on an image.

Aspects of the methods involve generating mathematical functions based on the data that is captured in an image using a detection component. For example, in some embodiments, the data from an image can be processed and transformed into a function that can be analyzed and manipulated mathematically using standard techniques. In some embodiments, an image is analyzed by determining the average gray-scale value of a column of pixels at each x coordinate, and the resulting data is converted into a function, or curve, that mathematically represents a signal from which the data was obtained. Once generated, the function can be analyzed or manipulated mathematically to determine its characteristics. In some embodiments, a plurality of pixel positions is plotted as a function of time to generate a time-based function representing, e.g., a change in the minimum value of an SPR signal as a function of time.

In some embodiments, a function can be analyzed to determine a minimum value or a maximum value using standard techniques. For example, in some embodiments, a first and/or second derivative of a function can be determined and used to calculate a relative minimum or relative maximum of the function. In some embodiments, a function can be smoothed using standard techniques, thereby reducing or diminishing noise in the data.

Aspects of the methods involve analyzing a function that is derived from an SPR signal in order to identify a pixel position corresponding to a minimum value of the function. The minimum value of the function corresponds to a reflectivity minimum of an SPR signal, and can be used in analyzing a sample (e.g., determining the concentration of an analyte in the sample).

Aspects of the methods involve analyzing a function that is derived from a critical angle signal in order to identify a pixel position corresponding to a maximum value of the function. The pixel position corresponding to the maximum value of the function can be used to determine the critical angle of the sensor.

In some embodiments, aspects of the methods involve analyzing data that is obtained from a reference feature. In some embodiments, the reference feature is an opto-mechanical reference (OMR) feature, and the data that is obtained from the OMR is one or more pixel positions from a reference signal that is generated by the OMR. For example, in some embodiments, an OMR creates a reference signal that can be analyzed to determine one or more parameters of a sample. In certain embodiments, a reference signal created by an OMR can be used as a fixed reference signal against which changes in an SPR minimum value (e.g., the number of pixels by which the SPR minimum value is moved, or shifted) can be measured when a sensing surface of a sensor is contacted with a sample, or is contacted with a plurality of different samples (e.g., an air sample and a water sample, a reference fluid sample and a blood sample, etc.). In certain embodiments, a reference signal created by an OMR can be used as a fixed reference signal that can be compared across different sample types (e.g., air and water, air and tear film, water and blood, etc.). In some embodiments, a reference feature is a data value obtained from one or more SPR signals, or one or more critical angle signals. For example, in some embodiments, a sensing surface of a sensor is contacted with a reference medium, and one or more SPR signals are generated. A pixel position corresponding to a minimum value of the one or more SPR signals, or a comparison of such minimum values, can be used as a reference feature. In some embodiments, one or more critical angle signals are generated from a sensor, and a pixel position corresponding to a maximum value of the one or more critical angle signals, or a comparison of such maximum values, can be used as a reference feature.

Aspects of the methods involve comparing pixel positions corresponding to various features of the above-described mathematical functions. For example, in some embodiments, a method involves comparing a pixel position of a minimum value of a function derived from a first SPR signal to the pixel position of a minimum value of a function derived from a second SPR signal to determine an SPR delta pixel value. The SPR delta pixel value represents the distance between the minimum values of the first and second SPR signals. In some embodiments, the methods involve comparing a pixel position of a maximum value of a function derived from a first critical angle signal to the pixel position of a maximum value of a function derived from a second critical angle signal to determine a critical angle delta pixel value. The critical angle delta pixel value represents the distance between the maximum values of the first and second critical angle signals.

In some embodiments, the methods involve mathematically manipulating a delta pixel value to account for one or more external conditions that can impact the operation of a subject sensor. For example, in some embodiments, the methods involve multiplying or dividing a delta pixel value by a correction factor in order to account for an external condition. As reviewed above, in some embodiments, a subject system can include an environmental analysis component that can be used to measure one or more characteristics of the environment in which the sensor is operating.

In some embodiments, the methods involve verifying a quality parameter of a sensor. For example, in some embodiments, one or more characteristics of a signal that is generated by a sensor is evaluated to determine whether the sensor is of sufficient quality for use. In some embodiments, one or more characteristics of an SPR signal is evaluated to determine whether the sensor is of sufficient quality for use. In certain embodiments, a contrast value, shape, or dimension (e.g., height, width, or depth) of an SPR signal (or a data set or function derived therefrom) is evaluated to determine if the sensor is of sufficient quality for use. In some embodiments, one or more characteristics of a critical angle signal is evaluated to determine whether the sensor is of sufficient quality for use. In certain embodiments, a contrast value, shape, or dimension (e.g., height, width, or depth) of a critical angle signal (or a data set or function derived therefrom) is evaluated to determine if the sensor is of sufficient quality for use. In some embodiments, the methods can be used to verify whether a sensor has, e.g., a sufficient thickness of a semitransparent film and/or adhesion layer on the sensing surface, or a sufficient purity of a material in the semitransparent film and/or adhesion layer.

Aspects of the methods involve comparing one or more data values (e.g., one or more delta pixel values, one or more corrected delta pixel values) to a calibration data set in order to determine a characteristic of a sample (e.g., a concentration of an analyte in the sample). In some embodiments, a system can include a plurality of calibration data sets that can be used for different purposes. In some embodiments, a system includes a calibration data set that includes analyte concentration values as a function of delta pixel values, and the methods involve comparing a delta pixel value to the calibration data set to determine the concentration of an analyte in a sample. In some embodiments, a system includes a calibration data set that includes quality parameter values, and the methods involve comparing one or more characteristics of a signal that is generated by a sensor to the calibration data set to determine whether the sensor is of sufficient quality for use. In some embodiments, a system includes a calibration data set that includes correction factors for various external environment parameters, and the methods involve comparing a measured external environment parameter to the calibration data set to determine an appropriate correction factor, and then mathematically manipulating a delta pixel value to apply the correction factor.

In some embodiments, a method involves determining a reference value (e.g., an SPR reference value, a critical angle reference value, an OMR reference value) and a test value (e.g., an SPR test value), and comparing the test value to the reference value. In certain embodiments, the difference between the test value and the reference value is then compared to a calibration data set to quantify a result (e.g., to provide a quantitative determination of the concentration of an analyte in a solution).

Methods in accordance with embodiments of the invention include both qualitative and quantitative detection. As such, in some embodiments, the subject methods involve determining whether an analyte is present in a sample at a concentration that is above or below a target, or threshold, concentration. In some embodiments, the subject methods involve quantitatively determining the concentration of an analyte in a sample. In certain embodiments, the methods involving comparing a result obtained from a sensor to one or more calibration values that can be used to quantitatively determine a concentration of an analyte in a sample.

In some embodiments, a method involves operably connecting a sensor to an optical chassis. In certain embodiments, a method involves removably coupling a sensor to an optical chassis, carrying out an analysis method, as described herein, and then removing the sensor from the optical chassis. In some embodiments, the methods involve aseptically coupling a sensor to an optical chassis. In some embodiments, the methods involve aseptically de-coupling a sensor from an optical chassis.

Aspects of the methods involve the analysis of any suitable sample. In some embodiments, a sample is a gaseous or a liquid medium. In certain embodiments, a medium can be a calibration medium, having a known analyte concentration. For example, in some embodiments, the methods involve contacting a sensor with a medium having a known analyte concentration, directing one or more optical signals to interact with the sensing surface, and detecting one or more signals resulting therefrom (e.g., detecting an SPR signal or a critical angle signal). In some embodiments, a sample can be a reference medium (e.g., a medium against which a test medium or sample will be compared). In some embodiments, a reference medium can be air (e.g., the air in a room where the sensor is used). In some embodiments, a reference medium can be a reference liquid (e.g., sterile water, containing a zero concentration of the analyte being tested for, or a calibration solution containing a known concentration of the analyte being tested for). In some embodiments, a sample is a liquid medium, e.g., water. In some embodiments, a sample can be a biological sample, as described above (e.g., blood). In some embodiments, the methods involve contacting a sensing surface of a sensor with a sample, and maintaining contact between the sample and the sensing surface while at least some of the method steps are carried out.

In certain embodiments, the methods involve measuring the concentration of two or more different species of analyte in a sample, e.g., measuring the concentration of IgG and IgM antibody isotypes in a sample that both bind to an antigen immobilized on the sensing surface of the sensor. In such embodiments, the methods involve contacting the sensor with the test solution, which contains both analyte species, and then contacting the sensor with a solution that inhibits the binding interaction of one of the species (e.g., the IgG isotype antibodies) with the antigen. After inhibiting one of the species from interacting with the sensor, another reading is taken, which represents binding of the other species with the antigen. The test signals obtained from these two different states (i.e., before and after binding inhibition of one of the species of analyte) can then be used to determine the relative concentration of each species in the sample.

In a preferred embodiment, a method involves contacting a sensing surface of a sensor comprising a first member of a binding pair immobilized thereon with a reference fluid and directing an optical signal having a first wavelength to interact with the sensing surface over a first range of incident angles to generate a first surface plasmon resonance (SPR) signal. An image of the first SPR signal is generated using the detection component, and the pixel position of a minimum value of the first SPR signal on the generated image is determined to generate an SPR reference value.

Next, the sensor is contacted with a test sample that contains the second member of the binding pair, and an optical signal having the first wavelength is directed to interact with the sensing surface over the second range of incident angles to generate a second SPR signal. A series of images of the second SPR signal is generated over a first time interval using the detection component, and a series of pixel positions that correspond to a minimum value of the second SPR signal is determined over the first time interval.

Next, a rate of change of the series of pixel positions that corresponds to the minimum value of the second SPR signal over the first time interval is determined, and a plateau value of the second SPR signal is determined based on the rate of change of the series of pixel positions that corresponds to the minimum value of the second SPR signal over the first time interval to generate an SPR test value. The SPR test value is then compared to the SPR reference value to detect the presence of the second member of the binding pair in the test sample.

In one preferred embodiment, a method further comprises directing an optical signal having a second wavelength to interact with the sensing surface over the first range of incident angles to generate a third SPR signal while the sensing surface is in contact with the reference fluid, and generating an image of the third SPR signal using the detection component.

Next, a pixel position of a minimum value of the third SPR signal is determined on the generated image, and the pixel position of the minimum value of the first SPR signal and the pixel position of the minimum value of the third SPR signal are combined to generate the SPR reference value.

In one preferred embodiment, a method further comprises directing an optical signal having a second wavelength to interact with the sensing surface over the second range of incident angles to generate a fourth SPR signal while the sensing surface is in contact with the test sample, and generating a series of images of the fourth SPR signal over a second time interval using the detection component.

Next, a series of pixel positions that corresponds to a minimum value of the fourth SPR signal is determined over the second time interval, and a rate of change of the series of pixel positions that corresponds to the minimum value of the fourth SPR signal over the second time interval is determined. A plateau value of the fourth SPR signal is then determined based on the rate of change of the series of pixel positions that corresponds to the minimum value of the fourth SPR signal over the second time interval, and the plateau value of the second SPR signal and the plateau value of the fourth SPR signal are combined to generate the SPR test value.

In one preferred embodiment, a method further comprises directing an optical signal having a first wavelength to interact with the sensing surface over the first range of incident angles to generate a first critical angle signal while the sensing surface is in contact with the reference fluid, and generating an image of the first critical angle signal using the detection component. Next, a pixel position of a maximum value of the first critical angle signal on the generated image is used to generate a critical angle reference value, which can be further applied to the calculation of the concentration of the analyte being tested for.

In one preferred embodiment, a method further comprises directing an optical signal having a second wavelength to interact with the sensing surface over the first range of incident angles to generate a second critical angle signal while the sensing surface is in contact with the reference fluid, and generating an image of the second critical angle signal using the detection component. Next, a pixel position of a maximum value of the second critical angle signal is determined on the generated image, and the pixel position of the maximum value of the first critical angle signal and the pixel position of the maximum value of the second critical angle signal are combined to generate the critical angle reference value, which can be further applied to the calculation of the concentration of the analyte being tested for.

In one preferred embodiment, a method further comprises determining a pixel position corresponding to an internal reference feature. In one preferred embodiment, the internal reference feature comprises an opto-mechanical reference feature.

In some embodiments, the first range of incident angles spans about 40 to 45 degrees. In one preferred embodiment, the first optical signal interacts with the sensing surface at an angle of about 42 degrees.

In some embodiments, the second range of incident angles spans about 62 to 67 degrees. In one preferred embodiment, the second optical signal interacts with the sensing surface at an angle of about 64 degrees.

In some embodiments, the methods involve capturing the images of the SPR signals in a single image frame. In certain embodiments, the images of the SPR signals and the images of the critical angle signals are captured in a single image frame.

In some embodiments, the methods involve comparing one or more generated values to a calibration data set. In certain embodiments, the methods further involve comparing one or more generated values to an external environment parameter to generate an external environment corrected value, and comparing the external environment corrected value to a calibration data set. In some embodiments, the external environment parameter is selected from the group comprising: temperature, pressure, humidity, light, environmental composition, or any combination thereof.

In a preferred embodiment, a method involves determining an antibody isotype response in a subject. This method involves contacting a sensing surface of a sensor, comprising an antigen immobilized thereon, with a reference fluid. Next, an optical signal having a first wavelength is directed to interact with the sensing surface over the first range of incident angles to generate a first surface plasmon resonance (SPR) signal. Next, an image of the first SPR signal is generated using the detection component, and a pixel position of a minimum value of the first SPR signal is determined on the generated image to generate an SPR reference value.

Next, the sensing surface is contacted with a sample from the subject, wherein the sample comprises a plurality of antibody isotypes that bind to the antigen. An optical signal having the first wavelength is directed to interact with the sensing surface over the second range of incident angles to generate a second SPR signal, and a series of images of the second SPR signal over a first time interval is generated using the detection component. Next, a series of pixel positions that correspond to a minimum value of the second SPR signal is determined over the first time interval. A rate of change of the series of pixel positions that corresponds to the minimum value of the second SPR signal is then determined over the first time interval, and a plateau value of the second SPR signal is determined based on the rate of change of the series of pixel positions that corresponds to the minimum value of the second SPR signal over the first time interval, which is used to generate a first SPR test value.

Next, the sensing surface is contacted with a stripping agent that removes at least one antibody isotype (e.g., an IgG stripping agent). An optical signal having the first wavelength is directed to interact with the sensing surface over the second range of incident angles to generate a third SPR signal. A series of images of the third SPR signal is generated over a second time interval using the detection component, and a series of pixel positions that correspond to a minimum value of the third SPR signal is determined over the second time interval.

Next, a rate of change of the series of pixel positions that corresponds to the minimum value of the third SPR signal is determined over the second time interval, and a plateau value of the third SPR signal is determined based on the rate of change of the series of pixel positions that corresponds to the minimum value of the third SPR signal over the second time interval, which is used to generate a second SPR test value. The first SPR test value, the second SPR test value, and the SPR reference value are then compared to determine the antibody isotype response in the subject.

In a preferred embodiment, a method involves determining a coronavirus exposure status in a patient. This method involves contacting a sensing surface of a sensor, comprising a coronavirus antigen immobilized thereon, with a reference fluid, and directing an optical signal having a first wavelength to interact with the sensing surface over a first range of incident angles to generate a first surface plasmon resonance (SPR) signal. An image of the first SPR signal is then generated using the detection component, and a pixel position of a minimum value of the first SPR signal is then determined on the generated image to generate an SPR reference value.

Then the sensing surface is contacted with a sample from the patient, wherein the sample comprises a plurality of IgG and IgM isotype antibodies that bind to the coronavirus antigen. An optical signal having the first wavelength is directed to interact with the sensing surface over a second range of incident angles to generate a second SPR signal. A series of images of the second SPR signal is generated over a first time interval using the detection component, and a series of pixel positions that correspond to a minimum value of the second SPR signal is determined over the first time interval. A rate of change of the series of pixel positions that corresponds to the minimum value of the second SPR signal is then determined over the first time interval, and a plateau value of the second SPR signal based on the rate of change of the series of pixel positions that corresponds to the minimum value of the second SPR signal over the first time interval is determined, and is used to generate a combined IgM IgG SPR test value.

The sensing surface is then contacted with an IgG stripping agent to remove IgG isotype antibodies. An optical signal having the first wavelength is then directed to interact with the sensing surface over the second range of incident angles to generate a third SPR signal. A series of images of the third SPR signal is generated over a second time interval using the detection component, and a series of pixel positions that correspond to a minimum value of the third SPR signal over the second time interval is determined.

A rate of change of the series of pixel positions that corresponds to the minimum value of the third SPR signal over the second time interval is then determined, and a plateau value of the third SPR signal based on the rate of change of the series of pixel positions that corresponds to the minimum value of the third SPR signal over the second time interval is then determined and used to generate an IgM SPR test value. Finally, the combined IgM IgG SPR test value, the IgM SPR test value, and the SPR reference value are compared to determine the coronavirus exposure status of the patient.

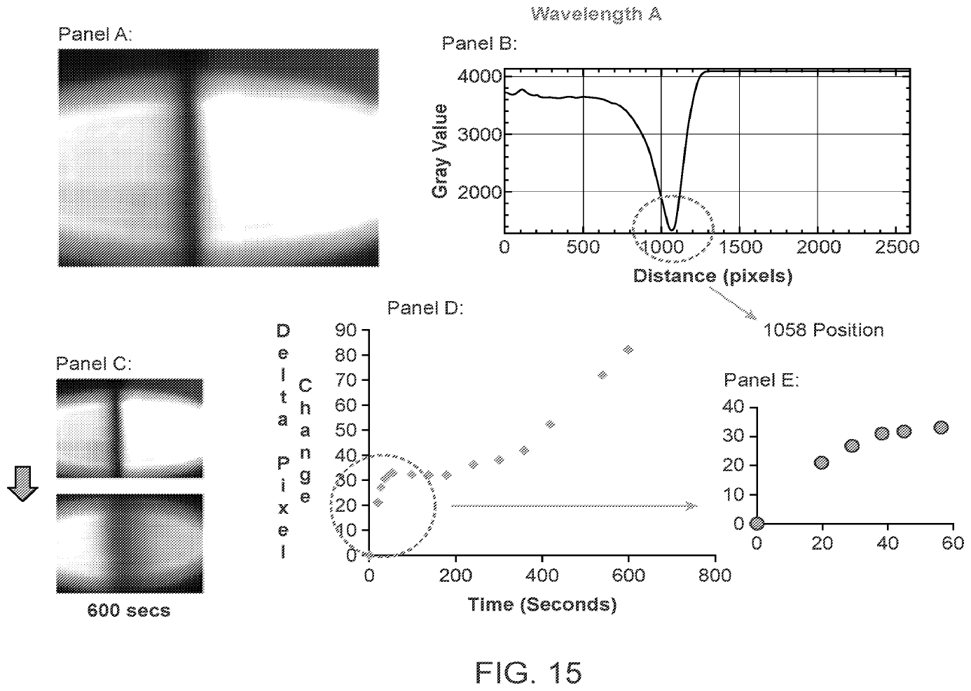

Turning now to FIG. 15, Panel A is an image of an SPR signal acquired with air as the reference medium in contact with the sensing surface of the sensor. Panel B is a graph of grey value as a function of pixel position for the optical signal shown in Panel A. Panel C provides two images of an SPR signal acquired at two different times, t=0 and t=600 seconds. Panel D is a graph showing the pixel position of the minimum value of the SPR signal shown in Panel C as a function of time after the sensing surface was contacted with a biological sample (e.g., a tear fluid). Panel E is a graph showing a close-up view of the pixel position of the minimum value of the SPR signal shown in Panel D over a time interval of 60 seconds and obtained using an optical signal having a wavelength of 855 nm, and corrected by subtracting the pixel position at t=0 seconds from the pixel position of the minimum value of the SPR signal measured at each indicated time point.

The following examples are provided to aid the understanding of the present invention, the true scope of which is set forth in the appended claims. It is understood that modifications can be made in the procedures set forth without departing from the spirit of the invention.

### EXAMPLES

### Example 1: Self-Calibrating Sensor Concept

A basic self-calibrating SPR sensor concept evolved from the illustrations in FIG. 6 and FIG. 7. FIG. 6 depicts a single piece injection molded sensor formed in an optical grade plastic. This one piece sensor concept was intended to utilize kinematic mounting features to constrain it in six degrees of freedom to assure each and every sensor was precisely and repeatably aligned to the optical chassis of the system. As shown in FIG. 7, the concept envisioned a sensor comprised of three segments—a base portion provides the precision kinematic mechanical interface to the optical chassis, an SPR prism portion with a gold (or protected silver) coated SPR sensing surface for obtaining an SPR signal from a sample, and finally, a “skirt” portion to provide the transition between the SPR prism portion and the base portion. The prism portion provides for self-calibration by implementing means for obtaining both an optical critical angle transition and an air SPR line, preferably at two separate wavelengths of approximately 850 nm and 950 nm, as well as another separate SPR line that was to appear when the gold coated sensor surface of the SPR prism was wetted by the tear fluid.

FIG. 8 illustrates a concept of an SPR sensor that uses ellipsoidal surfaces to image light from an LED source onto the sensing surface. As shown in FIG. 9, in order to be able to produce both an air SPR line and a tear (or water) SPR line, there must be light incident on the sensing surface at about 42.0° to produce the air SPR line and at approximately 64.4° to produce a tear SPR line. This is achieved by imaging light from a point source LED using an elliptical surface to relay an image of the LED onto the sensing surface (e.g., a gold coated sensing surface) of the transparent elliptically shaped reflector. The angles of incidence of the LED light on the internal elliptically shaped surface are such that total internal reflectance occurs for the LED light. Light reflected by the gold coated SPR sensing surface is then reflected back toward the point source LED by the left hand elliptically shaped inner surface and is intercepted by a beamsplitter that reflects returning light to an image sensor that detects the location of the SPR line. For the case of a rotationally symmetric ellipsoidal sensor, the SPR line is actually an SPR circle centered on the rotational axis of the ellipsoidal surface.

### Example 2: Analysis of Tear Fluid

A sensor comprising a sensing surface with a gold film was used to analyze a sample of tear fluid. The sensor was connected to a system, and the sensing surface was contacted with air as a reference medium. An optical signal having a wavelength of 855 nm was directed to interact with the sensing surface at an incident angle of approximately 42 degrees. The SPR signal from the sensing surface was detected using a detection component (FIG. 15, Panel A), and the pixel position corresponding to the minimum value of the SPR signal in air was determined (FIG. 15, Panel B).

Next, a sample of tear fluid was obtained from Ursa BioScience (Abingdon, MD) and a small volume of the sample was placed in contact with a sensing surface of the sensor. An optical signal having a wavelength of 855 nm was directed at the sensing surface at an incident angle of approximately 64 degrees. When the tear fluid was placed in contact with the sensing surface, an instantaneous change in the pixel position corresponding to the minimum value of the SPR signal was detected (FIG. 15, Panel D), relative to the pixel position corresponding to the minimum value of the SPR signal in air. The tear fluid was left in contact with the sensing surface for 600 seconds, and data was collected over this time interval. The pixel position corresponding to the minimum value of the SPR signal changed over time, eventually reaching a plateau value. FIG. 15, Panel E, shows a graph of the SPR delta pixel value, measured using an optical signal having a wavelength of 855 nm, and over a time interval of 60 seconds following contact of the sensing surface with the tear fluid. The graph in FIG. 15, Panel E has y-axis units of pixels and x-axis units of seconds. Each data point in the depicted graph was obtained by subtracting the pixel position corresponding to the minimum value of the SPR signal at t=0 from the pixel position corresponding to the minimum value of the SPR signal at each subsequent time point. A mathematical function was generated from the plotted data points depicted in FIG. 15, Panel E, and the function was then analyzed to determine the osmolarity of the tear fluid by comparison to a calibration data set.

### Example 3: Quantitative Determination of Anti-SARS-CoV-2 Spike Protein IgM and IgG Antibody Concentrations is a Blood Sample

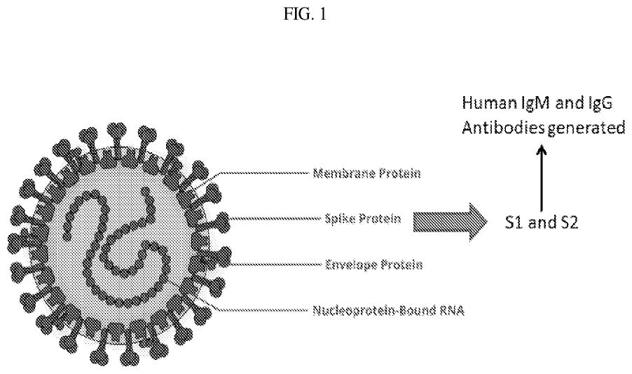

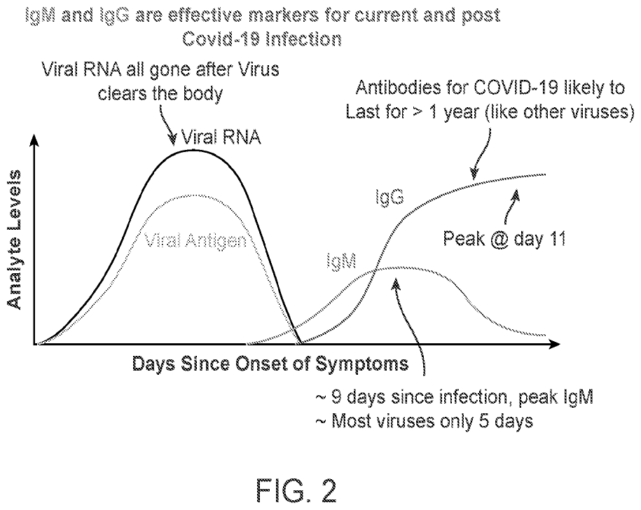

A sensor comprising a sensing surface with a gold film and a plurality of SARS-CoV-2 spike protein antigens immobilized thereon is used to detect IgM and IgG antibodies that bind to the spike protein in a blood sample, and the results are used to determine whether a patient has been recently infected, late stage infected, recovered, or never infected with SARS-CoV-2. As depicted in FIG. 1, several different viral proteins can serve as antigens that can be used to analyze an immune response in a patient, for example, a coronavirus spike protein. As depicted in FIG. 2, the relative concentrations of IgG and IgM antibodies that bind to a viral antigen can be used to judge the amount of time that has passed since viral infection occurred, and the resulting immune response of the patient.

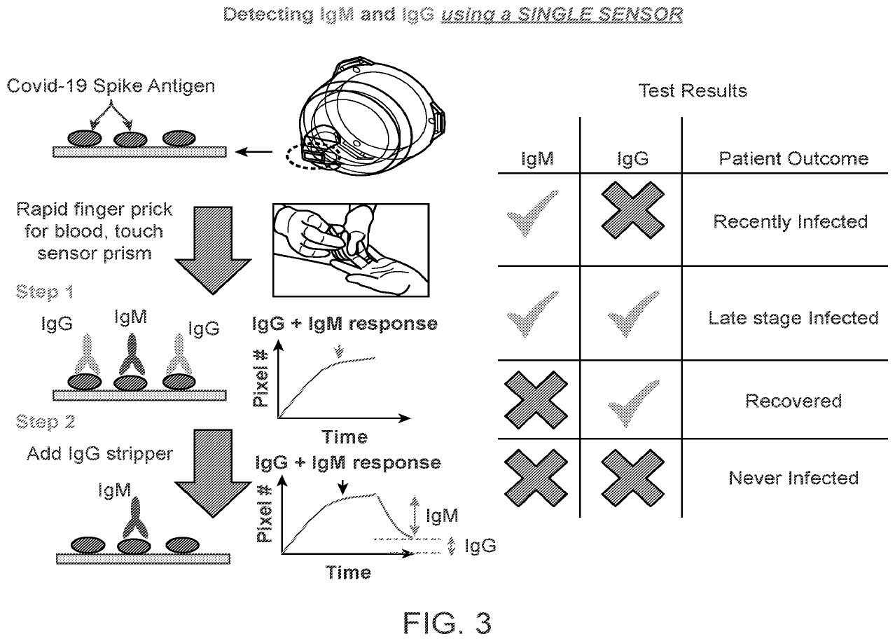

As depicted in FIG. 3, a sensor comprising a plurality of SARS-CoV-2 (COVID-19) spike protein antigens is used in the analysis. First, the sensor is self-calibrated by contacting the sensing surface with a reference medium (e.g., sterile water, or air). An optical signal having a first wavelength is directed to interact with the sensing surface over a first range of incident angles, selected depending on the refractive index of the reference medium. The SPR signal from the sensing surface is detected using the detection component, and the pixel position corresponding to the minimum value of the SPR signal is determined to generate an SPR reference value.

Next, a finger prick technique is used to draw blood from the patient's finger, and the sensing surface of the sensor is contacted with the blood. An optical signal having a first wavelength is directed at the sensing surface over a second range of incident angles, selected based on the refractive index of blood. As the antibodies in the patient's blood bind to the antigen on the sensing surface, the SPR signal changes with time. The pixel position corresponding to the minimum value of the SPR signal is detected over a time interval, and a plateau value is determined, which constitutes the SPR signal obtained from the combination of the IgG and IgM antibodies in the patient's blood.

Next, an IgG stripping agent is contacted to the sensor surface to interrupt binding of the IgG antibodies to the surface, thereby leaving behind only the IgM antibodies. The optical signal having the first wavelength is directed at the sensing surface over the second range of incident angles. As the IgG antibodies dissociate from the sensing surface, the SPR signal changes with time. The pixel position corresponding to the minimum value of the SPR signal is detected over a time interval, and a plateau value is determined, which constitutes the SPR signal obtained from only the IgM antibodies in the patient's blood.

The plateau values from the combined IgG IgM SPR signal, and from the IgM SPR signal, are compared to one another to determine the relative contribution of each species to the obtained SPR signals, and these values are then compared to SPR reference value and a calibration data set to determine a quantitative concentration of each analyte in the blood sample.

As depicted in FIG. 3, the concentration of the IgG and IgM species in the blood sample can then be used to determine the patient outcome, namely, whether the patient has been recently infected, is late stage infected, is recovered, or has never been infected.

### Example 4: Analysis of Donor Plasma

A sensor comprising a sensing surface with a gold film and a plurality of SARS-CoV-2 spike protein antigens immobilized thereon is used to detect IgG antibodies that bind to the spike protein in a blood sample, and the results are used to determine whether the donor plasma is suitable for use in convalescent plasma therapy, either for therapeutic or prophylactic purposes.

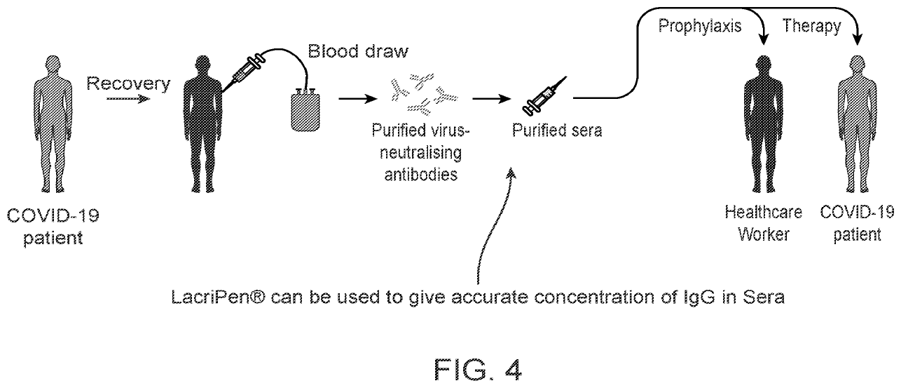

As depicted in FIG. 4, a plasma sample is obtained from a donor who has recovered from a viral infection (e.g., a SARS-CoV-2 infection, also known as COVID-19). A sensor comprising a plurality of SARS-CoV-2 (COVID-19) spike protein antigens is used in the analysis. First, the sensor is self-calibrated by contacting the sensing surface with a reference medium (e.g., sterile water, or air). An optical signal having a first wavelength is directed to interact with the sensing surface over a first range of incident angles, selected depending on the refractive index of the reference medium. The SPR signal from the sensing surface is detected using the detection component, and the pixel position corresponding to the minimum value of the SPR signal is determined to generate an SPR reference value.

Next, the sensing surface of the sensor is contacted with a sample of the donated plasma. An optical signal having a first wavelength is directed at the sensing surface over a second range of incident angles, selected based on the refractive index of plasma. As the antibodies in the plasma bind to the antigen on the sensing surface, the SPR signal changes with time. The pixel position corresponding to the minimum value of the SPR signal is detected over a time interval, and a plateau value is determined, which constitutes the SPR signal obtained from the antibodies in the donated plasma that bind to the viral antigen.

The plateau value is compared to the SPR reference value, and then to a calibration data set to determine a quantitative concentration of the antibodies in the donated plasma. The resulting concentration can then be used to determine whether the donated plasma is suitable for use in therapeutic or prophylactic applications.

Although the foregoing invention has been described in some detail by way of illustration and example for purposes of clarity of understanding, it is readily apparent to those of ordinary skill in the art in light of the teachings of this invention that certain changes and modifications can be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

1. A sensor comprising:
a sensing surface comprising a coated region, wherein the coated region comprises a first member of a binding pair immobilized thereon; and
wherein the sensor is configured to:
direct a first optical signal to interact with the sensing surface over a first range of incident angles; and
direct a second optical signal to interact with the sensing surface over a second range of incident angles,
wherein the first range of incident angles is different from the second range of incident angles.

2. The sensor according to claim 1, wherein the sensor comprises a plurality of facets.

3. The sensor according to claim 1, wherein the sensor has a frustoconical, concave shape.

4. The sensor according to claim 3, wherein the sensor comprises a plurality of facets on an internal surface and a plurality of facets on an external surface.

5. The sensor according to claim 4, wherein the sensor comprises 2 facets on the internal surface and 4 facets on the external surface.

6. The sensor according to claim 1, wherein the sensing surface is disposed on a central portion of the sensor.

7. The sensor according to claim 1, wherein the sensing surface further comprises a non-coated region.

8. The sensor according to claim 1, wherein the coated region comprises a semitransparent film that comprises a noble metal.

9. The sensor according to claim 8, wherein the noble metal is selected from the group consisting of: gold, silver, aluminum, platinum, palladium, or any combination thereof.

10. The sensor according to claim 8, wherein the semitransparent film has a thickness that ranges from about 0.5 nm to about 200 nm.

11. The sensor according to claim 10, wherein the semitransparent film has a thickness of about 45 to about 50 nm.

12. The sensor according to claim 1, wherein the coated region comprises an adhesion layer that is disposed between the sensor and the semitransparent film.

13. The sensor according to claim 12, wherein the adhesion layer has a thickness that ranges from about 0.5 nm to about 200 nm.

14. The sensor according to claim 12, wherein the adhesion layer has a thickness that ranges from about 45 nm to about 50 nm.

15. The sensor according to claim 12, wherein the adhesion layer comprises a material selected from the group consisting of: chromium, titanium dioxide, titanium monoxide, silicon dioxide, silicon monoxide, or any combination thereof.

16. The sensor according to claim 12, wherein the adhesion layer has an index of refraction that is different from an index of refraction of the sensor.

17. The sensor according to claim 1, wherein the first range of incident angles spans about 40 to 45 degrees.

18. The sensor according to claim 17, wherein the sensor is configured to direct the first optical signal to interact with the sensing surface at an angle of about 42 degrees.

19. The sensor according to claim 1, wherein the second range of incident angles spans about 62 to 67 degrees.

20. The sensor according to claim 19, where the sensor is configured to direct the second optical signal to interact with the sensing surface at an angle of about 64 degrees.

21. The sensor according to claim 1, wherein the binding pair is an antigen-antibody binding pair, and wherein the first member of the binding pair is the antigen.

22. The sensor according to claim 1, wherein the binding pair is an antigen-antibody binding pair, and wherein the first member of the binding pair is the antibody.

23. The sensor according to claim 21, wherein the antigen is a viral protein antigen.

24. The sensor according to claim 23, wherein the viral protein antigen is selected from the group consisting of: a viral membrane protein, a viral envelop protein, or a viral nucleoprotein.

25. The sensor according to claim 23, wherein the viral protein antigen is a coronavirus spike protein.

26. The sensor according to claim 25, wherein the coronavirus spike protein is a SARS-CoV-2 spike protein.

27. The sensor according to claim 26, wherein the SARS-CoV-2 spike protein is an S1 or an S2 subunit protein.

28. A system comprising:
(i) a sensor according to any one of claims 1-27; and
(ii) an optical chassis comprising:
an optical signal generating component;
a detection component;
a processor;
a controller; and
a computer-readable medium comprising instructions that, when executed by the processor, cause the controller to:
direct an optical signal having a first wavelength to interact with the sensing surface over the first range of incident angles to generate a first surface plasmon resonance (SPR) signal;
generate an image of the first SPR signal using the detection component;
determine a pixel position of a minimum value of the first SPR signal on the generated image to generate an SPR reference value;
direct an optical signal having the first wavelength to interact with the sensing surface over the second range of incident angles to generate a second SPR signal;
generate a series of images of the second SPR signal over a first time interval using the detection component;
determine a series of pixel positions that correspond to a minimum value of the second SPR signal over the first time interval;
determine a rate of change of the series of pixel positions that corresponds to the minimum value of the second SPR signal over the first time interval;
determine a plateau value of the second SPR signal based on the rate of change of the series of pixel positions that corresponds to the minimum value of the second SPR signal over the first time interval to generate an SPR test value; and
compare the SPR test value to the SPR reference value.

29. The system according to claim 28, wherein the computer-readable medium further comprises instructions that, when executed by the processor, cause the controller to:
direct an optical signal having a second wavelength to interact with the sensing surface over the first range of incident angles to generate a third SPR signal;
generate an image of the third SPR signal using the detection component;
determine a pixel position of a minimum value of the third SPR signal on the generated image; and
combine the pixel position of the minimum value of the first SPR signal and the pixel position of the minimum value of the third SPR signal to generate the SPR reference value.

30. The system according to claim 28 or 29, wherein the computer-readable medium further comprises instructions that, when executed by the processor, cause the controller to:
direct an optical signal having a second wavelength to interact with the sensing surface over the second range of incident angles to generate a fourth SPR signal;
generate a series of images of the fourth SPR signal over a second time interval using the detection component;
determine a series of pixel positions that corresponds to a minimum value of the fourth SPR signal over the second time interval;
determine a rate of change of the series of pixel positions that corresponds to the minimum value of the fourth SPR signal over the second time interval;
determine a plateau value of the fourth SPR signal based on the rate of change of the series of pixel positions that corresponds to the minimum value of the fourth SPR signal over the second time interval; and
combine the plateau value of the second SPR signal and the plateau value of the fourth SPR signal to generate the SPR test value.

31. The system according to any one of claims 28-30, wherein the computer-readable medium further comprises instructions that, when executed by the processor, cause the controller to:
direct an optical signal having a first wavelength to interact with the sensing surface over the first range of incident angles to generate a first critical angle signal;
generate an image of the first critical angle signal using the detection component; and
determine a pixel position of a maximum value of the first critical angle signal on the generated image to generate a critical angle reference value.

32. The system according to claim 31, wherein the computer-readable medium further comprises instructions that, when executed by the processor, cause the controller to:
direct an optical signal having a second wavelength to interact with the sensing surface over the first range of incident angles to generate a second critical angle signal;
generate an image of the second critical angle signal using the detection component;
determine a pixel position of a maximum value of the second critical angle signal on the generated image; and
combine the pixel position of the maximum value of the first critical angle signal and the pixel position of the maximum value of the second critical angle signal to generate the critical angle reference value.

33. The system according to claim 31 or 32, wherein the sensor comprises a coated region and a non-coated region, and wherein the first and second critical angle signals are generated from the non-coated region.

34. The system according to any one of claims 28-33, wherein the computer-readable medium further comprises instructions that, when executed by the processor, cause the controller to determine a pixel position corresponding to an internal reference feature.

35. The system according to claim 34, wherein the internal reference comprises an opto-mechanical reference feature.

36. The system according to any one of claims 28-35, wherein the computer-readable medium further comprises instructions that, when executed by the processor, cause the controller to compare one or more generated values to a calibration data set.

37. The system according to claim 28, wherein the first range of incident angles spans about 40 to 45 degrees.

38. The system according to claim 37, wherein the sensor is configured to direct the first optical signal to interact with the sensing surface at an angle of about 42 degrees.

39. The system according to claim 28, wherein the second range of incident angles spans about 62 to 67 degrees.

40. The system according to claim 39, where the sensor is configured to direct the second optical signal to interact with the sensing surface at an angle of about 64 degrees.

41. The system according to any one of claims 28-40, wherein the optical signal generating component comprises a laser or a light emitting diode (LED).

42. The system according to claim 41, wherein the laser or the LED emits visible or infrared light.

43. The system according to claim 42, wherein the laser or the LED emits light having a wavelength that ranges from about 400 to about 1,000 nm.

44. The system according to claim 43, wherein the laser or the LED is configured to emit light having a wavelength of about 855 nm.

45. The system according to claim 44, wherein the laser or the LED is configured to emit light having a wavelength of about 950 nm.

46. The system according to any one of claims 28-45, wherein the optical chassis further comprises one or more optical signal manipulation components.

47. The system according to any one of claims 28-46, wherein the detection component comprises an image sensor.

48. The system according to claim 47, wherein the image sensor is a charge coupled device (CCD) camera or a scientific complementary metal-oxide semiconductor (sCMOS) camera.

49. The system according to claim 47, wherein the image sensor is an active pixel sensor (APS).

50. The system according to any one of claims 28-49, further comprising a plurality of retention fixtures that are configured to removably couple the sensor to the optical chassis.

51. The system according to any one of claims 28-50, further comprising an alignment component that is configured to align the sensor with the optical chassis.

52. The system according to claim 51, wherein the alignment component comprises a tapered centering component.

53. The system according to any one of claims 28-52, further comprising a plurality of kinematic mounting components.

54. The system according to any one of claims 28-53, wherein the sensor is configured to be removably coupled to the optical chassis.

55. The system according to any one of claims 28-54, wherein the system is a benchtop system.

56. The system according to any one of claims 28-54, wherein the system is a hand-held system.

57. A method for detecting the presence of a second member of a binding pair in a test sample, the method comprising:
contacting a sensing surface of a system according to any one of claims 28-56 with a reference fluid;
directing an optical signal having a first wavelength to interact with the sensing surface over the first range of incident angles to generate a first surface plasmon resonance (SPR) signal;
generating an image of the first SPR signal using the detection component;
determining a pixel position of a minimum value of the first SPR signal on the generated image to generate an SPR reference value;
contacting the sensing surface with a test sample;
directing an optical signal having the first wavelength to interact with the sensing surface over the second range of incident angles to generate a second SPR signal;
generating a series of images of the second SPR signal over a first time interval using the detection component;
determining a series of pixel positions that correspond to a minimum value of the second SPR signal over the first time interval;
determining a rate of change of the series of pixel positions that corresponds to the minimum value of the second SPR signal over the first time interval;
determining a plateau value of the second SPR signal based on the rate of change of the series of pixel positions that corresponds to the minimum value of the second SPR signal over the first time interval to generate an SPR test value; and
comparing the SPR test value to the SPR reference value to detect the presence of the second member of the binding pair in the test sample.

58. The method according to claim 57, further comprising:
directing an optical signal having a second wavelength to interact with the sensing surface over the first range of incident angles to generate a third SPR signal while the sensing surface is in contact with the reference fluid;
generating an image of the third SPR signal using the detection component;
determining a pixel position of a minimum value of the third SPR signal on the generated image; and
combining the pixel position of the minimum value of the first SPR signal and the pixel position of the minimum value of the third SPR signal to generate the SPR reference value.

59. The method according to claim 57 or 58, further comprising:
directing an optical signal having a second wavelength to interact with the sensing surface over the second range of incident angles to generate a fourth SPR signal while the sensing surface is in contact with the test sample;
generating a series of images of the fourth SPR signal over a second time interval using the detection component;
determining a series of pixel positions that corresponds to a minimum value of the fourth SPR signal over the second time interval;
determining a rate of change of the series of pixel positions that corresponds to the minimum value of the fourth SPR signal over the second time interval;
determining a plateau value of the fourth SPR signal based on the rate of change of the series of pixel positions that corresponds to the minimum value of the fourth SPR signal over the second time interval; and
combining the plateau value of the second SPR signal and the plateau value of the fourth SPR signal to generate the SPR test value.

60. The method according to any one of claims 57-59, further comprising:
directing an optical signal having a first wavelength to interact with the sensing surface over the first range of incident angles to generate a first critical angle signal while the sensing surface is in contact with the reference fluid;
generating an image of the first critical angle signal using the detection component; and
determining a pixel position of a maximum value of the first critical angle signal on the generated image to generate a critical angle reference value.

61. The method according to claim 60, further comprising:
directing an optical signal having a second wavelength to interact with the sensing surface over the first range of incident angles to generate a second critical angle signal while the sensing surface is in contact with the reference fluid;
generating an image of the second critical angle signal using the detection component;
determining a pixel position of a maximum value of the second critical angle signal on the generated image; and
combining the pixel position of the maximum value of the first critical angle signal and the pixel position of the maximum value of the second critical angle signal to generate the critical angle reference value.

62. The method according to any one of claims 57-61, further comprising determining a pixel position corresponding to an internal reference feature.

63. The method according to claim 62, wherein the internal reference feature comprises an opto-mechanical reference feature.

64. The method according to claim 57, wherein the first range of incident angles spans about to 45 degrees.

65. The method according to claim 64, wherein the sensor is configured to direct the first optical signal to interact with the sensing surface at an angle of about 42 degrees.

66. The method according to claim 57, wherein the second range of incident angles spans about 62 to 67 degrees.

67. The method according to claim 66, where the sensor is configured to direct the second optical signal to interact with the sensing surface at an angle of about 64 degrees.

68. The method according to any one of claims 57-67, wherein the images of the SPR signals are captured in a single image frame.

69. The method according to claim 68, wherein the images of the SPR signals and the images of the critical angle signals are captured in a single image frame.

70. The method according to any one of claims 57-69, further comprising comparing one or more generated values to a calibration data set.

71. The method according to any one of claims 57-70, further comprising:
comparing one or more generated values to an external environment parameter to generate an external environment corrected value; and
comparing the external environment corrected value to a calibration data set.

72. The method according to claim 71, wherein the external environment parameter is selected from the group comprising: temperature, pressure, humidity, light, environmental composition, or any combination thereof.

73. The method according to any one of claims 57-72, wherein the optical signals having a first and a second wavelength are directed to interact with the sensing surface simultaneously.

74. The method according to any one of claims 57-72, wherein the optical signals having a first and second wavelength are directed to interact with the sensing surface in a gated manner.

75. The method according to any one of claims 57-74, wherein the calibration data set is stored in a read-only memory of a processor of the system.

76. The method according to any one of claims 57-75, wherein the sample is a biological sample.

77. The method according to claim 76, wherein the biological sample comprises blood.

78. The method according to any one of claims 57-77, wherein the reference fluid is air.

79. The method according to any one of claims 57-78, wherein the first time interval ranges from about 0.001 seconds to about 90 seconds.

80. The method according to any one of claims 57-78, wherein the second time interval ranges from about 0.001 seconds to about 90 seconds.

81. A method for determining an antibody isotype response in a subject, the method comprising:
contacting a sensing surface of a system according to any one of claims 28-56 with a reference fluid;
directing an optical signal having a first wavelength to interact with the sensing surface over the first range of incident angles to generate a first surface plasmon resonance (SPR) signal;
generating an image of the first SPR signal using the detection component;
determining a pixel position of a minimum value of the first SPR signal on the generated image to generate an SPR reference value;
contacting the sensing surface with a sample from the subject, wherein the binding pair is an antigen-antibody binding pair, wherein the first member of the binding pair is the antigen, and wherein the sample comprises a plurality of antibody isotypes that bind to the antigen;
directing an optical signal having the first wavelength to interact with the sensing surface over the second range of incident angles to generate a second SPR signal;
generating a series of images of the second SPR signal over a first time interval using the detection component;
determining a series of pixel positions that correspond to a minimum value of the second SPR signal over the first time interval;
determining a rate of change of the series of pixel positions that corresponds to the minimum value of the second SPR signal over the first time interval;
determining a plateau value of the second SPR signal based on the rate of change of the series of pixel positions that corresponds to the minimum value of the second SPR signal over the first time interval to generate a first SPR test value;
contacting the sensing surface with a stripping agent that removes at least one antibody isotype;
directing an optical signal having the first wavelength to interact with the sensing surface over the second range of incident angles to generate a third SPR signal;
generating a series of images of the third SPR signal over a second time interval using the detection component;
determining a series of pixel positions that correspond to a minimum value of the third SPR signal over the second time interval;
determining a rate of change of the series of pixel positions that corresponds to the minimum value of the third SPR signal over the second time interval;
determining a plateau value of the third SPR signal based on the rate of change of the series of pixel positions that corresponds to the minimum value of the third SPR signal over the second time interval to generate a second SPR test value;
comparing the first SPR test value, the second SPR test value, and the SPR reference value to determine the antibody isotype response in the subject.

82. A method for determining a coronavirus exposure status in a patient, the method comprising:
contacting a sensing surface of a system according to any one of claims 28-56 with a reference fluid;
directing an optical signal having a first wavelength to interact with the sensing surface over the first range of incident angles to generate a first surface plasmon resonance (SPR) signal;
generating an image of the first SPR signal using the detection component;
determining a pixel position of a minimum value of the first SPR signal on the generated image to generate an SPR reference value;
contacting the sensing surface with a sample from the patient, wherein the binding pair is an antigen-antibody binding pair, wherein the first member of the binding pair is a coronavirus antigen, and wherein the sample comprises a plurality of IgG and IgM isotype antibodies that bind to the coronavirus antigen;
directing an optical signal having the first wavelength to interact with the sensing surface over the second range of incident angles to generate a second SPR signal;
generating a series of images of the second SPR signal over a first time interval using the detection component;
determining a series of pixel positions that correspond to a minimum value of the second SPR signal over the first time interval;
determining a rate of change of the series of pixel positions that corresponds to the minimum value of the second SPR signal over the first time interval;
determining a plateau value of the second SPR signal based on the rate of change of the series of pixel positions that corresponds to the minimum value of the second SPR signal over the first time interval to generate a combined IgM IgG SPR test value;
contacting the sensing surface with an IgG stripping agent;
directing an optical signal having the first wavelength to interact with the sensing surface over the second range of incident angles to generate a third SPR signal;
generating a series of images of the third SPR signal over a second time interval using the detection component;
determining a series of pixel positions that correspond to a minimum value of the third SPR signal over the second time interval;
determining a rate of change of the series of pixel positions that corresponds to the minimum value of the third SPR signal over the second time interval;
determining a plateau value of the third SPR signal based on the rate of change of the series of pixel positions that corresponds to the minimum value of the third SPR signal over the second time interval to generate an IgM SPR test value;
comparing the combined IgM IgG SPR test value, the IgM SPR test value, and the SPR reference value to determine the coronavirus exposure status of the patient.
